(19) United States
(12) Patent Application Publication
(10) Pub. No.: US 2024/0003870 A1
(43) Pub. Date: Jan. 4, 2024

(54) METHOD OF SELECTING T CELLS WITH IMPROVED ANTI-CANCER ACTIVITY

(71) Applicants: METAFORA BIOSYSTEMS, Paris (FR); CENTRE NATIONAL DE LA RECHERCHE SCIENTIFIQUE, Paris (FR); UNIVERSITÉ DE MONTPELLIER, Montpellier (FR); THE UNITED STATES OF AMERICA ,ASREPRESENTED BY THE SECRETARY, DEPARTMENT OF HEALTH AND HUMAN SERVICE, Rockville, MD (US)

(72) Inventors: Naomi TAYLOR, Washington, DC (US); Marie Charlotte Chantal POUZOLLES, Washington, DC (US); Valérie DARDALHON, Montpellier (FR); Vincent PETIT, Paris (FR)

(73) Assignees: METAFORA BIOSYSTEMS, Paris (FR); CENTRE NATIONAL DE LA RECHERCHE SCIENTIFIQUE, Paris (FR); UNIVERSITÉ DE MONTPELLIER, Montpellier (FR); THE UNITED STATES OF AMERICA ,ASREPRESENTED BY THE SECRETARY, DEPARTMENT OF HEALTH AND HUMAN SERVICE, Rockville, MD (US)

(21) Appl. No.: 18/251,751

(22) PCT Filed: Nov. 5, 2021
(86) PCT No.: PCT/EP2021/080796

§ 371 Date: May 4, 2023

(30) Foreign Application Priority Data
Nov. 5, 2020 (EP) 20306327.6

(51) Int. Cl. G01N 33/50; A61K 35/17; A61P 35/00

(52) U.S. Cl. CPC G01N 33/5023; A61K 35/17; A61P 35/00

(57) ABSTRACT

A method of selecting T cells with improved anti-cancer activity, the method including: a) quantifying glucose transporter 1 (GLUT1) expression level at the cell surface of a population of T cells by using a GLUT1 ligand, b) selecting T cells having a low GLUT1 expression level, wherein the T cells having a low GLUT1 expression level have improved anti-cancer activity. Also, a population of T cells with improved anti-cancer activity for use in the treatment of cancer, to the use of a GLUT1 ligand for selecting T cells with improved anti-cancer activity, and to the use of GLUT1 as a biomarker of the anti-cancer therapeutic efficacy of T cells.

### FIELD OF INVENTION

The present invention pertains to the field of cell therapy for the treatment of cancer.

In particular, the present invention relates to a method of selecting T cells with improved anti-cancer activity, said method comprising a) quantifying glucose transporter 1 (GLUT1) expression level at the cell surface of a population of T cells by using a GLUT1 ligand, b) selecting T cells having a low GLUT1 expression level, wherein said T cells having a low GLUT1 expression level have improved anti-cancer activity. The present invention also relates to a population of T cells with improved anti-cancer activity for use in the treatment of cancer, to the use of a GLUT1 ligand for selecting T cells with improved anti-cancer activity, and to the use of GLUT1 as a biomarker of the anti-cancer therapeutic efficacy of T cells.

### BACKGROUND OF INVENTION

Much progress has been made in the development of T cell therapies for the treatment of cancer. However, optimizing the persistence and function of adoptively transferred T cells remains a challenge, resulting in the search and development of novel strategies.

One important aspect impacting T cell proliferation and function is the host environment. The activation and effector function of adoptively-transferred T lymphocytes are associated with increased energetic and biosynthetic demands, generally secured by augmented nutrient entry and utilization. Indeed, TCR-stimulated upregulation of glucose as well as amino acid transporters at the cell surface might play a significant role for optimal T cell proliferation and effector functions. Importantly though, distinct T lymphocyte subsets exhibit disparate metabolic profiles: T effector cells are highly glycolytic, and even lipogenic, while suppressive regulatory T cells (Tregs) display a mixed metabolism with increased levels of lipid oxidation. In this regard, it is interesting to note that Treg, but not Teff, generation is maintained under conditions where nutrient transport, such as glucose, glutamine or leucine transport, is limiting (Macintyre et al., Cell Metab 20, 61-72 (2014); Nakaya et al. Immunity 40, 692-705 (2014); Sinclair et al., Nat Immunol 14, 500-508 (2013).

Furthermore, the potential of an engineered anti-tumor T cell to respond to tumor antigens is often negatively modulated by the metabolic environment of the tumor. This environment is conditioned by nutrient composition, “waste” products, oxygen concentration, pH, temperature and physical forces, amongst others. Indeed, the dysregulated growth of cancer cells can directly influence the extracellular environment and multiple studies have found that the metabolic phenotype of a tumor governs the ensuing immune response (reviewed in Mayers & Vander Heiden, Cancer research 77, 3131-3134 (2017)).

Given the complex cellular, signaling and metabolic situation in tumor lesions, the inventors tried to elucidate the metabolic characteristics that promote optimal in vivo T cell anti-tumoral effector functions, in particular in the “hostile” tumor tissue that consumes high levels of nutrients and is often anaerobic. To this end, the inventors specifically evaluated the expression of nutrient transporters, for instance by using the receptor binding domain (RBD) technology, and analyzed the in vivo anti-tumoral properties of subpopulations of T cells with distinct nutrient transporter profiles.

The inventors surprisingly showed that T cells having a low GLUT-1 expression level at the cell surface exhibited improved anti-cancer activity. In particular, they showed that T cells having low GLUT-1 expression level at the cell surface adoptively transferred to tumor-bearing mice were capable of decreasing tumor burden better than T cells having high GLUT-1 expression level at the cell surface.

The inventors thus showed that T cells selected on the basis of low GLUT1 expression levels at the cell surface exhibited increased anti-cancer activity.

### SUMMARY

The invention relates to a method of selecting T cells with improved anti-cancer activity, said method comprising:
a) quantifying glucose transporter 1 (GLUT1) expression level at the cell surface of a population of T cells by using a GLUT1 ligand,
b) selecting T cells having a low GLUT1 expression level, wherein said T cells having a low GLUT1 expression level have improved anti-cancer activity.

According to a first embodiment, said method comprises:
a0) contacting a population of T cells expressing GLUT1 at their cell surface, or susceptible to express GLUT1 at their cell surface, with a GLUT1 ligand,
a1) detecting and/or quantifying the binding of said GLUT1 ligand to GLUT1 at the cell surface of the T cells,
a2) quantifying GLUT1 expression level at the cell surface of the T cells, b) selecting T cells having a low GLUT1 expression level, and c) optionally, isolating the selected T cells having a low GLUT1 expression level.

According to a second embodiment, said T cells having a low GLUT1 expression level corresponds to the at most 50%, 40%, 30%, 20%, 10% or 5% fraction with the lowest GLUT1 expression level within the total GLUT1+T cell population.

According to a third embodiment, quantifying GLUT1 expression level at the cell surface of the T cells is done by flow cytometry.

The invention also concerns a population of T cells with improved anti-cancer activity, for use in the treatment of cancer, wherein said T cells are selected by the method of the invention.

The invention further relates to the use of a glucose transporter 1 (GLUT1) ligand for selecting T cells with improved anti-cancer activity.

In some embodiments, said GLUT1 ligand is labeled.

In some embodiments, said GLUT1 ligand comprises a receptor binding domain (RBD) derived from the soluble part of an envelope glycoprotein of a primate T-lymphotropic virus (PTLV), or comprises an antibody or an antigen-binding fragment thereof.

In some embodiments, said GLUT1 ligand comprises a receptor binding domain (RBD) derived from the soluble part of an envelope glycoprotein of human T-cell leukemia virus type 1 (HTLV-1), T-cell leukemia virus type 2 (HTLV-2), T-cell leukemia virus type 3 (HTLV-3), T-cell leukemia virus type 4 (HTLV-4), simian T-cell leukemia virus type 1 (STLV-1), simian T-cell leukemia virus type 2 (STLV-2), simian T-cell leukemia virus type 3 (STLV-3), or simian T-cell leukemia virus type 5 (STLV-5).

In some embodiments, said GLUT1 ligand comprises a receptor binding domain (RBD) derived from the soluble part of an envelope glycoprotein of T-cell leukemia virus type 2 (HTLV-2).

In some embodiments, said receptor binding domain (RBD) comprises or consists of the amino acid sequence SEQ ID NO: 15.

The invention also concerns the use of glucose transporter 1 (GLUT1) as a biomarker of the anti-cancer therapeutic efficacy of T cells.

In some embodiments, said T cells are selected from the group consisting of conventional CD4⁺ T cells, conventional CD8⁺ T cells, γδ T cells and double negative (DN) T cells.

In some embodiments, said T cells are chimeric antigen receptor (CAR) T cells.

In some embodiments, said cancer is a blood cancer or a solid tumor.

### DETAILED DESCRIPTION

The present invention relates to a method, preferably an in vitro method, of selecting T cells with improved anti-cancer activity, said method comprising:
a) quantifying glucose transporter 1 (GLUT1) expression level at the cell surface of a population of T cells by using a GLUT1 ligand,
b) selecting T cells having a low GLUT1 expression level, wherein said T cells having a low GLUT1 expression level have improved anti-cancer activity.

GLUT1 is a cell surface transporter of glucose, and more precisely a glucose importer. As used herein, “GLUT1” refers to a glucose importer expressed by metazoans, in particular by humans, used as receptor by viruses, in particular by Human T-cell Leukemia viruses (HTLV). In one embodiment, GLUT1 is human GLUT1 (accession number NP_006507.2, SEQ ID NO: 1). In one embodiment GLUT1 comprises or consists of an amino acid sequence presenting a sequence identity of at least 70% with SEQ ID NO: 1, preferably a sequence identity of at least 75, 80, 85, 90, 91, 92, 93, 94, 95, 96, 97, 98, 99% or more with SEQ ID NO: 1. In one embodiment, GLUT1 comprises or consists of a fragment of SEQ ID NO: 1, preferably a fragment of at least about 100 amino acids, more preferably of at least about 150, 200, 250, 300, 350, 400 or 450 amino acids.

In one embodiment, the method of the invention comprises determining, measuring or quantifying the expression level of GLUT1.

As used herein, the term “expression” may refer alternatively to the transcription of the slc2a1 gene coding for GLUT1 (i.e. expression of the RNA) or to the translation (i.e. expression of the protein) of GLUT1, or to the presence of GLUT1 at the surface of the cell. Methods for determining, measuring or quantifying the expression level are well-known from the skilled artisan, and include, without limitation, determining the transcriptome (in an embodiment wherein expression relates to transcription of slc2a1 or GLUT1) or proteome (in an embodiment wherein expression relates to translation of GLUT1) of a cell.

In one embodiment, the terms “determining”, “measuring” or “quantifying” the expression level of GLUT1 are equivalent. “Quantifying the expression level of GLUT1” herein means quantifying GLUT1 present at the cell surface. Quantifying the expression level of GLUT1 present at the cell surface may comprise detecting GLUT1 at the cell surface. Methods for analyzing the presence of a protein at the cell surface are well-known to the skilled artisan. In an embodiment, determining, measuring or quantifying the expression level of GLUT1 is performed by flow cytometry or Fluorescence Activated Cell Sorting (FACS). In another embodiment, determining, measuring or quantifying the expression level of GLUT1 is performed by bead-based assay, e.g. magnetic bead-based assay.

In one embodiment, determining, measuring or quantifying the expression level of GLUT1 corresponds to detecting and/or quantifying the binding of a ligand to GLUT1 present at the cell surface.

Thus, another object of the invention is a method of selecting T cells with improved anti-cancer activity, said method comprising:
a0) contacting a population of T cells expressing GLUT1 at their cell surface, or susceptible to express GLUT1 at their cell surface, with a GLUT1 ligand,
a1) detecting and/or quantifying the binding of said GLUT1 ligand to GLUT1 at the cell surface of the T cells,
a2) quantifying GLUT1 expression level at the cell surface of the T cells, b) selecting T cells having a low GLUT1 expression level, and c) optionally, isolating the selected T cells having a low GLUT1 expression level.

The term “ligand” refers to any substance that binds, specifically binds, or forms a complex with another molecule. Typical ligands include, but are not limited to, small molecules such as e.g. proteins, polypeptides, peptides, peptidomimetic compounds and other organic and inorganic small molecule compounds.

Preferably, the GLUT1 ligand is a molecule that interacts with GLUT1 at the cell surface, and the method of the invention comprises detecting and/or quantifying a complex formed between said ligand and GLUT1.

The expression “that interacts with GLUT1 at the cell surface” means that the ligand is liable to recognize the GLUT1 receptor present on the surface of the cell. In one embodiment, a ligand that interacts with GLUT1 at the cell surface will thus form a complex with said GLUT1 at the cell surface, which complex may be detected by a method as hereinabove described.

In one embodiment, the ligand binds to GLUT1. In one embodiment, the ligand specifically binds to GLUT1.

The expression “specifically binds to”, as used herein, refers to the binding specificity and affinity of a molecule or a domain thereof for a particular target or epitope, or a domain thereof, even in the presence of a heterogeneous population of other proteins and biological molecules. Thus, in one embodiment, under designated assay conditions, the ligand described in the invention binds preferentially to its target and does not bind in a significant amount to other components present in a test sample or subject. In one embodiment, such a ligand shows high affinity binding to its target with an equilibrium dissociation constant equal or below 1×10⁻⁶ M (e.g., at least 0.5×10⁻⁶, 1×10⁻⁷, 1×10⁻⁸, 1×10⁻⁹, 1×10⁻¹⁰ and less). Standard assays to evaluate the binding ability of two biological molecules are known in the art, including for example, ELISAs, Western blots, RIAs and flow cytometry. The binding kinetics (e.g., binding affinity) of the molecules also can be assessed by standard assays known in the art, such as by Biacore analysis.

The expression “detecting and/or quantifying the binding of a ligand, e.g. a GLUT1 ligand, to GLUT1” means that, when GLUT1 is present at the cell surface, a complex is formed between GLUT1 and the ligand. That complex can be detected if the ligand is, for example but not limited to, covalently coupled to a detectable label or molecule such as an antibody constant fragment (Fc) or a fluorescent compound. The complex can also be detected if the ligand is tagged with different means well known to the person skilled in the art. The use of the ligand therefore allows on the one hand the identification and detection of GLUT1 at the cell surface depending on the ligand used, and on the other hand the quantification of the complex formed.

In one embodiment, the GLUT1 ligand is labeled.

In one embodiment, the GLUT1 ligand is labeled with a fluorescent label. Examples of fluorescent label include, but are not limited to, fluorescent organic dyes, quantum dots and fluorescent proteins. These ligands may be useful as optical imaging probes.

Examples of fluorescent organic dyes include, but are not limited to, commercial Alexa Fluor® dyes, fluorescein, rhodamine, or Cy® dyes (such as Cy3, Cy3.5, Cy5, Cy5.5, Cy7, and Cy7.5).

Examples of fluorescent proteins include, but are not limited to, BFP, CFP, GFP, EGFP, mCherry, tdTomato, mPlum, mStrawberry, J-Red, DS-Red, mOrange, mCitrine, Venus, Ypet, YFP, Emerald, and the like. Another example of fluorescent protein is phycoerythrin. The fluorescent protein may be fused to the ligand by techniques of molecular cloning well known in the art. Alternatively, the fluorescent protein may be chemically conjugated to the ligand by techniques well known in the art.

In one embodiment, the GLUT1 ligand is labeled with a peptidic tag.

Examples of peptidic tags include, but are not limited to, an antibody crystallizable region (Fc), Enzyme (alkaline phosphatase or horseradish peroxidase), Hemagglutinin tag, Poly Arginine tag, Poly Histidine tag, Myc tag, Strep tag, S-tag, HAT tag, 3× Flag tag, Calmodulin-Binding Peptide tag, SBP tag, Chitin Binding Domain tag, GST tag, Maltose-Binding Protein tag, Fluorescent peptidic tag, T7 tag, V5 tag, X-press tag and the like. The peptidic tag may be fused to the ligand by techniques of molecular cloning well known in the art or covalently attached to the ligand.

In one embodiment, the GLUT1 ligand labeled as described herein is a fusion protein comprising a GLUT1 ligand (as described herein) fused to a detection tag, such as, for example, a fluorescent protein such as GFP, RFP, BFP, YFP and related proteins, or a

Fc fragment. In one embodiment the GLUT1 ligand is fused to a Fc fragment or a fluorescent protein. In one embodiment, the GLUT1 ligand labeled as described herein, comprises a GLUT1 ligand (as described herein) chemically conjugated to phycoerythrin.

The amino acid sequence SEQ ID NO: 2 is a non-limiting example of a GFP sequence. Examples of Fc fragments include, but are not limited to, rabbit Fc fragment (amino acid sequence SEQ ID NO: 3), mouse Fc fragment (amino acid sequence SEQ ID NO: 4).

In one embodiment, detecting and/or quantifying the binding is conducted by flow cytometry, or Fluorescence Activated Cell Sorting (FACS). In another embodiment, detecting and/or quantifying the binding is performed by bead-based assay, e.g. magnetic bead-based assay.

In an aspect of the invention, the ligand comprises a receptor binding domain derived from the soluble part of a glycoprotein of an enveloped virus that interacts with GLUT1 at the cell surface. Preferably, the ligand is soluble, i.e. it does not comprise a transmembrane domain, and is therefore not anchored to a membrane.

The expression “derived from the soluble part of an envelope glycoprotein of a virus” means that the ligand is a fragment of a glycoprotein of a viral envelope and can be obtained, for example, by cloning or gene synthesis. The term “glycoprotein” is to be understood as meaning an envelope glycoprotein, a coat glycoprotein or a fusion glycoprotein, wherein the term “glycoprotein” refers to a protein containing oligosaccharide chains covalently attached to polypeptide side-chains.

The receptor binding domain derived from the soluble part of a glycoprotein of an enveloped virus may also be an unglycosylated peptide or polypeptide, i.e. it may be derived from the unglycosylated form of the soluble part of a glycoprotein of an enveloped virus.

In one embodiment, said virus is a retrovirus, preferably a deltaretrovirus, such as e.g. a primate T cell leukemia virus (PTLV). Examples of primate T cell leukemia viruses (PTLV) include human T cell leukemia viruses (HTLVs) and simian T cell leukemia viruses (STLVs).

The deltaretroviruses encode an envelope glycoprotein present in mature retrovirus virions. The term “envelope protein” (“Env”, encoded by the env gene) refers to a protein synthesized as a single polyprotein, in the form of a propeptide, which is subsequently cleaved in the Golgi apparatus by a furin peptidase into two components: the amino terminal surface envelope protein (“SU protein”) and the carboxy terminal transmembrane envelope protein (“TM protein”). The Env protein is glycosylated. The SU protein is responsible for binding with the host receptor. It comprises a receptor-binding domain (RBD) liable to interact with host cell membrane receptors, and a domain of interaction with the TM domain The TM protein, located inside the lipid bilayer anchors the SU protein to the surface of viral particles. The TM protein mediates the membrane fusion reaction with the host cell.

In an embodiment, the receptor binding domain comprised in the GLUT1 ligand is isolated or derived from the soluble part of an envelope glycoprotein of a human T-cell leukemia virus.

In an embodiment, the receptor binding domain comprises or consists of the total surface envelope protein (SU) of a human T-cell leukemia virus.

In an embodiment, the receptor binding domain in a fragment of the surface envelope protein (SU) of a human T-cell leukemia virus.

In an embodiment, the receptor binding domain comprised in the GLUT1 ligand is isolated or derived from the soluble part of an envelope glycoprotein of human T-cell leukemia virus type 1 (HTLV-1).

In an embodiment, the receptor binding domain comprises or consists of the total surface envelope protein (SU) of human T-cell leukemia virus type 1 (HTLV-1), and preferably has a sequence comprising or consisting of the amino acid sequence SEQ ID NO: 5, SEQ ID NO: 32 or a fragment or variant thereof.

In an embodiment, the receptor binding domain is a fragment of the surface envelope protein (SU) of human T-cell leukemia virus type 1 (HTLV-1), and preferably has a sequence comprising or consisting of the amino acid sequence SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 33, SEQ ID NO: 34, or a fragment or variant thereof.

In an embodiment, the receptor binding domain comprised in the GLUT1 ligand is isolated or derived from the soluble part of an envelope glycoprotein of human T-cell leukemia virus type 2 (HTLV-2).

In an embodiment, the receptor binding domain comprises or consists of the total surface envelope protein (SU) of human T-cell leukemia virus type 2 (HTLV-2), and preferably has a sequence comprising or consisting of the amino acid sequence SEQ ID NO: 13, SEQ ID NO: 35, or a fragment or variant thereof.

In an embodiment, the receptor binding domain is a fragment of the surface envelope protein (SU) of human T-cell leukemia virus type 2 (HTLV-2), and preferably has a sequence comprising or consisting of the amino acid sequence SEQ ID NO: 14, SEQ ID NO: 15, SEQ ID NO: 16, SEQ ID NO: 36 or SEQ ID NO: 37, or a fragment or variant thereof.

In an embodiment, the receptor binding domain comprised in the GLUT1 ligand is isolated or derived from the soluble part of an envelope glycoprotein of human T-cell leukemia virus type 3 (HTLV-3).

In an embodiment, the receptor binding domain comprises or consists of the total surface envelope protein (SU) of human T-cell leukemia virus type 3 (HTLV-3), and preferably has a sequence comprising or consisting of the amino acid sequence SEQ ID NO: 17, SEQ ID NO: 38, or a fragment or variant thereof.

In an embodiment, the receptor binding domain is a fragment of the surface envelope protein (SU) of human T-cell leukemia virus type 3 (HTLV-3), and preferably has a sequence comprising or consisting of the amino acid sequence SEQ ID NO: 18, SEQ ID NO: 39, or a fragment or variant thereof.

In an embodiment, the receptor binding domain comprised in the GLUT1 ligand is isolated or derived from the soluble part of an envelope glycoprotein of human T-cell leukemia virus type 4 (HTLV-4).

In an embodiment, the receptor binding domain comprises or consists of the total surface envelope protein (SU) of human T-cell leukemia virus type 4 (HTLV-4), and preferably has a sequence comprising or consisting of the amino acid sequence SEQ ID NO: 19, SEQ ID NO: 40 or a fragment or variant thereof.

In an embodiment, the receptor binding domain is a fragment of the surface envelope protein (SU) of human T-cell leukemia virus type 4 (HTLV-4), and preferably has a sequence comprising or consisting of the amino acid sequence SEQ ID NO: 20 or SEQ ID NO: 21, SEQ ID NO: 41, SEQ ID NO: 42, or a fragment or variant thereof.

In an embodiment, the receptor binding domain comprised in the GLUT1 ligand is isolated or derived from the soluble part of an envelope glycoprotein of simian T-cell leukemia virus type 1 (STLV-1).

In an embodiment, the receptor binding domain comprises or consists of the total surface envelope protein (SU) of simian T-cell leukemia virus type 1 (STLV-1), and preferably has a sequence comprising or consisting of the amino acid sequence SEQ ID NO: 22, SEQ ID NO: 43 or a fragment or variant thereof.

In an embodiment, the receptor binding domain is a fragment of the surface envelope protein (SU) of simian T-cell leukemia virus type 1 (STLV-1), and preferably has a sequence comprising or consisting of the amino acid sequence SEQ ID NO: 23, SEQ ID NO: 44, or a fragment or variant thereof.

In an embodiment, the receptor binding domain comprised in the GLUT1 ligand is isolated or derived from the soluble part of an envelope glycoprotein of simian T-cell leukemia virus type 2 (STLV-2).

In an embodiment, the receptor binding domain comprises or consists of the total surface envelope protein (SU) of simian T-cell leukemia virus type 2 (STLV-2), and preferably has a sequence comprising or consisting of the amino acid sequence SEQ ID NO: 24, SEQ ID NO: 45, or a fragment or variant thereof.

In an embodiment, the receptor binding domain is a fragment of the surface envelope protein (SU) of simian T-cell leukemia virus type 2 (STLV-2), and preferably has a sequence comprising or consisting of the amino acid sequence SEQ ID NO: 25, SEQ ID NO: 46, or a fragment or variant thereof.

In an embodiment, the receptor binding domain comprised in the GLUT1 ligand is isolated or derived from the soluble part of an envelope glycoprotein of simian T-cell leukemia virus type 3 (STLV-3).

In an embodiment, the receptor binding domain comprises or consists of the total surface envelope protein (SU) of simian T-cell leukemia virus type 3 (STLV-3), and preferably has a sequence comprising or consisting of the amino acid sequence SEQ ID NO: 26, SEQ ID NO: 47, or a fragment or variant thereof.

In an embodiment, the receptor binding domain is a fragment of the surface envelope protein (SU) of simian T-cell leukemia virus type 3 (STLV-3), and preferably has a sequence comprising or consisting of the amino acid sequence SEQ ID NO: 27, SEQ ID NO: 48, or a fragment or variant thereof.

In an embodiment, the receptor binding domain comprised in the GLUT1 ligand is isolated or derived from the soluble part of an envelope glycoprotein of simian T-cell leukemia virus type 5 (STLV-5).

In an embodiment, the receptor binding domain comprises or consists of the total surface envelope protein (SU) of simian T-cell leukemia virus type 5 (STLV-5), and preferably has a sequence comprising or consisting of the amino acid sequence SEQ ID NO: 28, SEQ ID NO: 49, or a fragment or variant thereof.

In an embodiment, the receptor binding domain is a fragment of the surface envelope protein (SU) of simian T-cell leukemia virus type 3 (STLV-3).

Preferably, the receptor binding domain is isolated or derived from the soluble part of an envelope glycoprotein of T-cell leukemia virus type 2 (HTLV-2) and has a sequence comprising or consisting of the amino acid sequence SEQ ID NO: 15, herein referred to as HTLV2.RBD or H2.RBD.

The receptor binding domain comprised in the GLUT1 ligand may consist of the total RBD or of a fragment or a variant thereof.

By “fragment” of a reference sequence is meant herein a sequence constituted by a chain of consecutive amino acids of a reference sequence and whose size is smaller than the size of the reference sequence. In the context of the invention, the fragments may for example have a size between 6 and 200, 6 and 185, 6 and 175, 6 and 150, 6 and 125, 6 and 100, 6 and 75, 6 and 50, 6 and 25, 6 and 15, 6 and 10 amino acids, or a size of between 6 and 185, 10 and 185, 25 and 185, 50 and 185, 75 and 185, 100 and 185, 125 and 185, 150 and 185, 175 and 185 amino acids.

The term “variant” herein has the same meaning as “homologue” or “derivative”. A “variant” is defined as comprising a sequence identical to at least 75%, preferably at least 80%, preferably at least 85%, more preferably at least 90%, even at least 95%, 96%, 97%, 98% or 99% of the reference sequence.

By “amino acid sequence having (for instance) at least 80% of identity with a reference sequence” is meant herein a sequence identical to the reference sequence but this sequence may comprise up to twenty mutations (substitutions, deletions and/or insertions) per each part of one hundred amino acids of the reference sequence. Therefore, for a reference sequence of 100 amino acids, a fragment of 80 amino acids and a sequence of 100 amino acids comprising 20 substitutions compared with the reference sequence are two examples of sequences having 80% sequence identity with the reference sequence.

Percentage of identity is generally determined using sequence analysis software (for example the Sequence Analysis Software Package of the Genetics Computer Group, University of Wisconsin Biotechnology Center, 1710 University Avenue, Madison, Wis. 53705). The amino acid sequences to be compared are aligned to obtain maximum percentage identity. For this purpose, it may be necessary to artificially add gaps in the sequence. The alignment can be performed manually or automatically. Automated alignment algorithms of nucleotide sequences are well known to persons skilled in the art and described for example in Altschul et al. (1997) Nucleic Acids Res. 25:3389 and implemented by softwares such as the Blast software. One algorithm which can be isolated is the Needleman-Wunsch algorithm for example (Needleman and Wunsch (1970) J Mol Biol. 48:443-53). Once optimal alignment has been achieved, the percentage identity is established by recording all the positions at which the amino acids of the two compared sequences are identical, compared with the total number of positions.

These variant sequences may differ from the reference sequence by substitution, deletion and/or insertion of one or more amino acids, at positions such that these modifications do not have any significant impact on the biological activity of the polypeptides. The substitutions may in particular correspond to conservative substitutions or to substitutions of natural amino acids by non-natural amino acids or pseudo amino acids.

In one particular embodiment, the sequence of the polypeptides differs from the reference sequence solely through the presence of conservative substitutions. Conservative substitutions are substitutions of amino acids of the same class, such as substitutions of amino acids with non-charged side chains (such as asparagine, glutamine, serine, cysteine, and tyrosine), of amino acids with basic side chains (such as lysine, arginine and histidine), of amino acids with acid side chains (such as aspartic acid and glutamic acid), of amino acids with non-polar side chains (such as alanine, valine, leucine, isoleucine, proline, phenylalanine, methionine and tryptophan).

As used herein, a “variant” of a reference protein exhibits the same biological activity as the reference polypeptide. In particular, a variant of a receptor binding domain is capable of binding to the same receptor, and a variant of a GLUT1 ligand is capable of binding to GLUT1.

When it is desired to alter the amino acid sequence of a polypeptide to create an equivalent, or even an improved, variant or portion of a ligand of the invention, one skilled in the art will typically change one or more of the codons of the encoding DNA sequence. For example, certain amino acids may be substituted by other amino acids in a protein structure without appreciable loss of its ability to bind cell surface nutrient transporters. Since it is the binding capacity and nature of a protein that defines that protein's biological functional activity, certain amino acid sequence substitutions can be made in a protein sequence, and, of course, its underlying DNA coding sequence, and nevertheless obtain a protein with similar properties. It is thus contemplated that various changes may be made in the peptide sequences, or corresponding DNA sequences that encode said peptides without appreciable loss of their biological utility or activity.

In a preferred embodiment, variant polypeptides differ from a reference sequence by substitution, deletion or addition of 1, 2, 3, 4, 5, 6, 7, 8, 9 or 10 amino acids.

In one embodiment, the receptor binding domain can be fused to an antibody constant fragment (such as, for example, Fc fragment from rabbit, mouse, human, monkey or other mammals), and/or chemically modified to add a fluorochrome, or a fluorescent compound (e.g. Cyanine dye, Alexa dye, Quantum dye, etc).

In one embodiment, the GLUT1 ligand comprises a receptor binding domain, or a fragment or variant of said receptor binding domain, fused to a detection tag or label, such as e.g. a Fc fragment or a GFP. Examples of a GFP protein include, but are not limited to the amino acid sequence SEQ ID NO: 2. Examples of Fc fragments include, but are not limited to, rabbit Fc fragment (such as e g amino acid sequence SEQ ID NO: 3, mouse Fc fragment (such as e g amino acid sequence SEQ ID NO: 4), human Fc fragment or monkey Fc fragment.

In some embodiments, the receptor binding domain is fused to the detection tag or label via a linker. Such a linker may be useful to prevent steric hindrances. In some embodiments, the linker has 4; 5; 6; 7; 8; 9; 10; 11; 12; 13; 14; 15; 16; 17; 18; 19; 20; 21; 22; 23; 24; 25; 26; 27; 28; 29; 30 amino acid residues. The linker sequence may be a naturally occurring sequence or a non-naturally occurring sequence. One useful group of linker sequences are linkers derived from the hinge region of heavy chain antibodies. Other examples are poly-alanine linker sequences. Further preferred examples of linker sequences are Gly/Ser linkers of different length, i.e. linkers consisting of one or more glycine(s) followed by one or more serine(s).

In an embodiment, the receptor binding domain comprised in the GLUT1 ligand is HTLV2.RBD fused to a GFP, for instance via a Gly/Ser linker (amino acid sequence SEQ ID NO: 29 or SEQ ID NO: 50). In an embodiment, the receptor binding domain comprised in the GLUT1 ligand is HTLV2.RBD fused to a rabbit Fc fragment, for instance via a Gly/Ser linker (amino acid sequence SEQ ID NO: 30 or SEQ ID NO: 51). In an embodiment, the receptor binding domain comprised in the GLUT1 ligand is HTLV2.RBD fused to a mouse Fc fragment, for instance via a Gly/Ser linker (amino acid sequence SEQ ID NO: 31 or SEQ ID NO: 52).

In an embodiment, the receptor binding domain comprised in the GLUT1 ligand is HTLV2.RBD fused to a human Fc fragment, for instance via a Gly/Ser linker.

In another embodiment, said ligand is an antibody or an antigen-binding fragment thereof. In particular, said ligand is an antigen-binding fragment specific for GLUT1, and the method of the invention comprises detecting and/or quantifying a complex formed between said antigen-binding fragment and GLUT1.

As used herein, the term “antigen-binding fragment” refers to a fragment of an antibody which has the ability to specifically interact with (e.g., by binding, steric hindrance, stabilizing/destabilizing, spatial distribution) an epitope of an antigen. The antigen-binding fragment may in particular comprise a portion of an intact antibody, preferably the antigen binding region or variable region of the intact antibody. Examples of antigen-binding fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen-binding fragment can also be incorporated into single domain antibodies, maxibodies, minibodies, nanobodies, intrabodies, diabodies, triabodies, tetrabodies, a v-NAR and a bis-scFv. Antigen binding fragments can also be grafted into scaffolds based on polypeptides such as a fibronectin type III. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of crosslinking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

In an embodiment, said ligand is an antibody. In particular, said ligand is an antibody specific for GLUT1, and the method of the invention comprises detecting and/or quantifying a complex formed between said antibody and GLUT1.

Antibodies specific for GLUT1 are well known in the art. Preferably, the antibody does not require permeabilization treatment for binding to GLUT1. Non-limiting examples of antibodies specific for GLUT1 includes e.g. the recombinant anti-glucose transporter GLUT1 antibody clone # EPR3915, available from Abcam under the reference ab209449, or the monoclonal mouse IgG2B clone # 202915, available from R&D Systems under the reference MAB1418.

In addition to determining, measuring or quantifying the expression level of GLUT1 level at the cell surface of T cells, the methods of the invention further comprise a step of selecting T cells having a low GLUT1 expression level.

The terms “positive”, “negative”, “low” and “high”, when referring to the expression level of a cell surface marker, are well known in the art.

The terms “expressing”, “positive”, or “+” and “not expressing”, “negative”, or “-” are well known in the art and refer to the expression level of a cell marker of interest, in that the expression level of the cell marker corresponding to “+” is high or intermediate or low (i.e. the cell marker is expressed or present at the cell surface), and the expression level of the cell marker corresponding to “−” is null (i.e. the cell marker is not expressed, or is absent, at the cell surface).

The terms “low” or “lo” or “lo/−” are well known in the art and refers to the expression level of the cell marker of interest, in that the expression level of the cell marker is low in comparison with the expression level of that cell marker in the total population of cells being analyzed as a whole. More particularly, the term “lo” refers to a distinct population of cells that express the cell marker at a lower level than one or more other distinct populations of cells.

The term “high” or “hi” or “bright” is well known in the art and refers to the expression level of the cell marker of interest, in that the expression level of the cell marker is high in comparison with the expression level of that cell marker in the total population of cells being analyzed as a whole. More particularly, the term “hi” refers to a distinct population of cells that express the cell marker at a higher level than one or more other distinct populations of cells.

As a non-limiting example, cells in the top 2, 3, 4, 5, 10 or 15% of staining intensity are designated “hi” with the remaining cells falling in the top half of the population categorized as being “intermediate”. Also, as a non-limiting example, among the “positive” (or “+”) cells, the cells falling below 50% of fluorescence intensity are designated as “lo” cells.

In some embodiments, determining, measuring or quantifying the expression level of GLUT1 is performed by flow cytometry or Fluorescence Activated Cell Sorting (FACS).

In some embodiments, determining, measuring or quantifying the expression level of GLUT1 comprises determining the percentage or fraction of cells from the cell population expressing GLUT1 (i.e., cells “+” for GLUT1), preferably by FACS.

The expression level of GLUT1 is determined by comparing the Fluorescence Intensity (FI) of a cell from the cell population stained with a fluorescent ligand specific for GLUT1 to the FI of cells from the same unstained cell population, or stained with a fluorescent antibody with an irrelevant specificity but with the same isotype, the same fluorescent probe and originated from the same species (referred to as isotype control), or stained with a fluorescent ligand with an irrelevant specificity, e.g. a ligand specific for a receptor which is absent at the cell surface of the population, with the same fluorescent probe (referred to as control ligand). Cells stained with a fluorescent antibody specific for GLUT1 and that show equivalent FI or a lower FI than the unstained cells, or the cells stained with the isotype control or with the control ligand, are considered as not expressing this marker and are designated “-” or “negative”. Cells stained with a fluorescent antibody specific for GLUT1 that show a FI value superior to the cells stained with the isotype control are considered as expressing GLUT1 and are designated “+” or “positive”.

In some embodiments, the methods of the invention comprise determining the percentage or fraction of cells from the cell population expressing GLUT1 at a lower level. In preferred embodiments, determining the percentage or fraction of cells from the cell population expressing GLUT1 at a lower level is performed by flow cytometry (FACS).

In some embodiments, the methods of the invention comprise selecting T cells having a low GLUT1 expression level, either concomitantly with or after quantifying the expression level of GLUT1 level at the cell surface of T cells. In some embodiments, selecting T cells having a low GLUT1 expression level is performed by flow cytometry (FACS).

In an embodiment, the selected T cells, or T cell populations, having a low GLUT1 expression level corresponds to the at most 90%, 80%, 70%, 60%, 50%, 40%, 30%, 20%, 15%, 10% or 5% fraction with the lowest GLUT1 expression level among the total GLUT1+T cell population.

In another embodiment, the T cells, or T cell subpopulations, having a low GLUT1 expression level (T cell subpopulations selected or to be selected) corresponds to the 90%, 80%, 70%, 60%, 50%, 40%, 30%, 20%, 15%, 10% or 5% fraction with the lowest GLUT1 expression level among the total GLUT1+T cell population.

In one embodiment, when selecting T cells having a low GLUT1 expression level is performed by FACS, T cells having a low GLUT1 expression level (selected or to be selected) are identified as those with the 15% lowest GLUT1 staining, within the total GLUT1+cell population.

Preferably, the T cells, or T cell populations, having a low GLUT1 expression level have an improved anti-cancer activity as compared to the total T cell population, or to T cells having a high GLUT1 expression.

According to certain embodiments, the GLUT1 levels measured at the cell surface of the T cells in the total population are divided into equal-sized groups by cut-off values referred to as “quantiles”, each group corresponding to a determined percentage of T cells in the total population. Examples of quantiles include, without being limited to, the median (defining 2 groups based on GLUT1 level, each comprising 50% of the T cells in the total population), the terciles or tertiles (defining 3 groups based on GLUT1 level, each comprising a third of the T cells in the total population), the quartiles (defining 4 groups based on GLUT1 level, each comprising 25% of the T cells in the total population), the quintiles (defining 5 groups based on GLUT1 level, each comprising 20% of the T cells in the total population) and the deciles (defining 10 groups based on GLUT1 level, each comprising 10% of the T cells in the total population).

“Quantiles” sometimes refers to the cut-off GLUT1 values below or above which falls a determined percentage of the T cells in the total population, based on GLUT1 level. Therefore, the subjects with a measured GLUT1 level below the first quantile are the subjects with the lowest GLUT1 levels, while the subjects with a measured GLUT1 level above the last quantile are the human subjects with the highest GLUT1 levels.

Additionally, the term “quantiles” may also sometimes refer to the group so defined by said cut-off value. Thus, applied to the present invention, the term “quantiles” may also refer to the groups of T cells defined by the cut-off GLUT1 value. For example, the P t decile may refer to the group of T cells corresponding to the lowest 10% of GLUT1 levels measured in the total population. It follows that a GLUT1 value that is in the 1s t decile is a GLUT1 value comprised in the lowest 10% of GLUT1 levels measured in the T cell population.

As used herein, the term “quantile” preferably refers to the group so defined by said cut-off value.

In an embodiment, the predetermined GLUT1 value is the first GLUT1 quartile also referred to as GLUT1 “Ql” in the total GLUT1+T cell population.

In an embodiment, the T cells, or T cell subpopulations, having a low GLUT1 expression level (T cell subpopulations selected or to be selected) correspond to the first GLUT1 expression quartile in the total GLUT1+T cell population.

In certain embodiments, the T cells, or T cell subpopulations, having a low GLUT1 expression level (T cell subpopulation selected or to be selected) correspond to the first GLUT1 expression median, tercile, quartile, quintile or decile in the total GLUT1+T cell population.

In one embodiment, the GLUT1 levels measured at the cell surface of the T cells in the total population are divided into groups corresponding to a given percentage of T cells in the total population. As mentioned above, the cut-off values (“quantiles”) so dividing the GLUT1 levels measured in the T cell populations are called “percentiles”.

Thus, in one embodiment, the predetermined GLUT1 value is the first GLUT1 percentile in the total T cell population.

In one embodiment, preferably after isolating the GLUT1+T cell subpopulations, selecting T cells having a low GLUT1 expression level is performed by using magnetic particles, such as e.g. beads. For instance, selecting T cells having a low GLUT1 expression level is performed by negative magnetic immuno-adherence using ligands directed against GLUT1 present on the cells, the T cells having a low GLUT1 expression level being the “unselected” cells.

For isolation of a desired population of cells by positive or negative selection, in particular for depleting T cells having a high GLUT1 expression by positive selection, or for recovering unbounded T cells having a low GLUT1 expression by negative selection, the concentration of cells and surface (e.g., particles such as beads) can be varied.

In certain embodiments, it may be desirable to significantly increase the volume in which the beads and cells are mixed together (i.e., decrease the concentration of cells), to reduce contact of cells and beads. Use of low cell concentrations may induce less efficient capture of T cells weakly expressing GLUT1, or expressing low levels of GLUT1 at their cell surface.

In one embodiment, the expression level is normalized, i.e. the expression level corresponds to a ratio between the expression of GLUT1 and the expression of another gene or protein. For instance, the other gene or protein used for normalization may be another nutrient transporter, a phenotypic marker such as CD45. CD2, CD3, CD4, CD8 etc., or an engineered receptor such as a chimeric antigen receptor or TCR.

In the present invention, two numeric values, in particular two expression levels, are considered as different if the first numeric value is higher (such as, for example, the first numeric value is about 20% higher than the second one, preferably is about 30, 40, 50, 70, 80, 90% or more higher than the second one) or lower than the second one (such as, for example, the second numeric value is about 20% lower than the second one, preferably is about 30, 40, 50, 60, 70, 80, 90% or more lower than the second one).

The cell of the invention is a T cell. It may be a mammalian T cell, preferably a human T cell, a T cell from a farm animal (e.g., a cow, pig, or horse), or a T cell from a pet (e.g., a cat or a dog).

In some embodiments, the T cell is a conventional cytotoxic T cell or a conventional helper T cell.

A T cell is a type of lymphocyte. T cells originate as precursor cells derived from bone marrow and migrate to the thymus gland where they differentiate into several distinct types of T cells.

In some embodiments, prior to selection of a subpopulation of T cells expressing low levels of GLUT1 as described herein, the cells are obtained from a subject. T cells can be obtained from a number of sources, including peripheral blood mononuclear cells (PBMC), bone marrow, lymph node tissue, cord blood, thymus tissue, tissue from a site of infection, ascites, pleural effusion, spleen tissue, and tumors. In certain embodiments, T cells can be obtained from a unit of blood collected from a subject using any number of techniques known to the skilled artisan, such as Ficoll™ separation, centrifugation through a PERCOLL™ gradient following red blood cell lysis and monocyte depletion, counterflow centrifugal elutriation, leukapheresis, and subsequent cell surface marker-based magnetic or flow cytometric isolation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, cells from the circulating blood of an individual are obtained by leukapheresis.

In some embodiments, cells collected by leukapheresis may be washed to remove the plasma fraction and to place the cells in an appropriate buffer or media for subsequent processing steps. In some embodiments of the invention, the cells are washed with phosphate buffered saline (PBS). In some embodiments, the wash solution lacks calcium and may lack magnesium or may lack many if not all divalent cations. After washing, the cells may be resuspended in any of a variety of biocompatible buffers, such as, for example, Ca²⁺-free, Mg²⁺-free PBS, PlasmaLyte A, or other saline solutions with or without buffer. Alternatively, the undesirable components of the leukapheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells can be further isolated by positive or negative selection techniques. For example, in some embodiments, T cells are isolated by incubation with anti-CD3/anti-CD28-conjugated beads, such as DYNABEADS® M-450 CD3/CD28 T, for a time period sufficient for positive selection of the desired T cells. By simply shortening or lengthening the time that T cells are allowed to bind to the anti-CD3/anti-CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention.

In some embodiments, it may be desirable to perform the selection procedure and use the “unselected” cells in the activation and expansion process. “Unselected” cells can also be subjected to further rounds of selection. Enrichment of a T cell population by negative selection can be accomplished with a combination of antibodies directed to surface markers unique to the negatively selected cells. One method is cell sorting and/or selection via negative magnetic immuno-adherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. Further, use of high cell concentrations allows more efficient capture of cells that may weakly express target antigens of interest, or from samples where there are many tumor cells present (i.e., leukemic blood, tumor tissue, etc.).

In some embodiments, the T cell is selected from the group consisting of a conventional CD4⁺T cell, a conventional CD8⁺T cell, a γδ T cell, and a double negative (DN) T cell.

In certain embodiments, the T cell is a conventional CD4³⁰ T cell, such as, for example, a naïve CD4³⁰ T cell, a helper CD4³⁰ T cell, a central memory CD4³⁰ T cell, an effector memory CD4³⁰ T cell, or an effector CD4³⁰ T cell.

In some embodiments, the T cell is a conventional CD⁸⁺ T cell, such as, for example, a naïve CD⁸⁺T cell, a cytotoxic CD⁸⁺ T cell, a central memory CD8⁺T cell, an effector memory CD8⁺T cell, or an effector CD8⁺T cell.

In some embodiments, the T cell is a DN T cell.

In some embodiments, the T cell is a γδ T cell. In some embodiments the T cell is a TEGγδ (T cell engineered to express a defined Gamma delta TCR).

In some embodiments, the T cell is a T cell engineered to express a defined chimeric antigen receptor (CAR) at the cell surface.

As used herein, the term “chimeric antigen receptor” (CAR) or “chimeric receptor” (CR) refers to one polypeptide or to a set of polypeptides, typically two in the simplest embodiments, which when in an immune cell, provides the cell with specificity for a target ligand and with intracellular signal generation. In some embodiments, the chimeric receptor is a chimeric fusion protein comprising the set of polypeptides. In some embodiments, the set of polypeptides include a dimerization switch that, upon the presence of a dimerization molecule, can couple the polypeptides to one another, e.g., can couple a ligand binding domain to an intracellular signaling domain.

In some embodiments, the “chimeric antigen receptor” (CAR) comprises:
(a) an extracellular binding domain with a determined specificity,
(b) optionally an extracellular hinge domain,
(c) a transmembrane domain, and
(d) an intracellular signaling domain comprising a T cell primary signaling domain and, optionally, a T cell costimulatory signaling domain.

As a recombinant, composite membrane receptor, the CAR recognizes a surface antigen on the targeted cancer cell by an antibody-derived extracellular domain and activates the engineered T cell by a T cell receptor (TCR) derived intracellular signaling domain Following interaction with the tumor antigen, the T cell is activated through a combination of different signaling moieties on the CAR which can include the CD28 costimulatory domain, or the costimulatory domains of the TNF-receptor family such as 4-1BB (CD137), OX40 (CD135), and ICOS, either individually or in combination.

In some embodiments, the T cell is a T cell engineered to express a defined T cell receptor (TCR) at the cell surface.

In some embodiments, the T cells with improved anti-cancer activity, which are selected by the method of the invention, are engineered T cells.

In an embodiment, the initial T cell population is an engineered T cell population and the method of selecting T cells with improved anti-cancer activity according to the invention is implemented on said already engineered T cells. In this embodiment, the T cells are firstly engineered and are subsequently submitted to the method of selecting T cells with improved anti-cancer activity according to the invention.

In another embodiment, the method of selecting T cells with improved anti-cancer activity according to the invention is implemented first, on a non-engineered T cell population, and the selected T cells are subsequently engineered.

In some embodiments, the T cell is a tumor-infiltrating lymphocyte (TIL).

In some embodiments, the T cell is an effector T cell. In certain embodiments, the effector T cell is a CD4³⁰ effector T cell. Examples of CD4³⁰ effector T cells include, but are not limited to, Th1 cells, Th2 cells, Th9 cells, Th17cells, Th22 cells, and CD⁴⁺T follicular helper (Tfh) cells. In some embodiments, the effector T cell is a CD8⁺ effector T cell. In one embodiment, the T cell is an effector γδ T cell. In one embodiment, the immune cell is an effector DN T cell.

The inventors surprisingly showed that adoptive transfer of GLUT1^{low} T cells in tumor-bearing NSG mice resulted in a substantially improved tumor rejection, as compared to adoptive transfer of GLUT1^{high} T cells.

They further showed that low tumor burden was associated with a higher percentage of T cells in the GLUT1^{low} T cells group than in the GLUT1^{high} T cells group at day 43 post adoptive transfer.

The inventors therefore surprisingly showed that the GLUT1^{low} T cell population adoptively transferred to tumor-bearing mice was capable of better decreasing tumor burden than the GLUT1 ^{high} T cell population or the total T cell population. Thus, T cells selected on the basis of low GLUT1 expression levels exhibited improved anti-cancer activity and effector function.

Accordingly, an object of the invention is a population of T cells with improved anti-cancer activity, for use in the treatment of cancer, wherein said T cells are selected by the selection method of the invention.

As used herein, the terms “treating” or “treatment” or “alleviation” refer to therapeutic treatment, excluding prophylactic or preventative measures; wherein the object is to slow down (lessen) the targeted pathologic condition or disorder. Those in need of treatment include those already with the disorder as well as those suspected to have the disorder. A subject is successfully “treated” for the targeted pathologic condition or disorder if, after receiving a therapeutic amount of the cells, cell population, composition, pharmaceutical composition or medicament according to the present invention, said subject shows observable and/or measurable reduction in or absence of one or more of the symptoms associated with the specific disease or condition, reduced morbidity and mortality, and/or improvement in quality of life issues. The above parameters for assessing successful treatment and improvement in the disease are readily measurable by routine procedures familiar to a physician.

Another aspect of the invention is the use of a GLUT1 ligand for selecting T cells with improved anti-cancer activity.

Said GLUT1 ligand is preferably as described in the present specification.

In some embodiments, the T cells are selected based on their GLUT1 expression, for instance as described in the present specification. In preferred embodiments, the selected T cells are GLUT1^{low} T cells.

A further aspect is the use of GLUT1 as a biomarker of the anti-cancer therapeutic efficacy of T cells.

As used herein, the “therapeutic efficacy” of a compound or medicament against a pathologic disease, condition or disorder in a subject in need thereof may for instance be disclosed if, after receiving a therapeutic amount of the compound or medicament, the subject shows observable and/or measurable reduction in, or absence of, one or more of the symptoms associated with the specific disease, condition or disorder, reduced morbidity and mortality, and/or improvement in quality of life issues. The above parameters for assessing successful treatment and improvement in the disease are readily measurable by routine procedures familiar to a physician.

In particular, in the case where the disease is cancer, an “anti-cancer therapeutic efficacy” or “anti-cancer activity” of a compound or medicament may for instance correspond to the capacity of said compound or medicament to reduce the size of a tumor or of metastasis, or to prevent tumor growth or metastasis development or spread.

It is also disclosed herein a method of treating cancer in a subject in need thereof, comprising:
selecting T cells with improved anti-cancer activity by the selection method according to the invention, and
administering a therapeutic amount of said T cells with improved anti-cancer activity to the subject.

In some embodiments, the T cells are selected based on their GLUT1 expression. Preferably, the selected T cells are GLUT1^{low} T cells.

As used herein, the term “subject” refers to a warm-blooded animal, preferably a mammal. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human. In one embodiment, a subject may be a “patient”, i.e., a subject who/which is awaiting the receipt of, or is receiving medical care or was/is/will be the object of a medical procedure, or is monitored for the development of a disease.

In some embodiments, the T cells are autologous cells. In some embodiments, the T cells are heterologous cells. In some embodiments, the T cells are allogenic cells.

A further aspect is the use of the T cells or the T cell population of the invention, for the manufacture of a medicament for the treatment of cancer.

Another aspect is a composition, a pharmaceutical composition, or a medicament comprising the T cells or the T cell population of the invention for use in the treatment of cancer.

As used herein, the term “cancer” has its general meaning in the art and in particular refers to a disease caused by an uncontrolled division of abnormal cells. The term “cancer” encompasses solid tumors and blood cancers, and encompasses both primary and metastatic cancers.

Examples of cancers include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus.

In one embodiment, said cancer is a tumor, such as, for example, a solid tumor.

In another embodiment, said cancer is a blood cancer. In another embodiment, said cancer is a hematologic malignancy. Examples of hematologic cancers include, but are not limited to, Hodgkin's disease, non-Hodgkin's lymphoma (malignant lymphoma) and blood cancers such as e.g. acute or chronic myeloid leukemia, acute lymphoblastic leukemia, chronic lymphocytic leukemia, myeloproliferative diseases, multiple myeloma, myelodysplastic syndrome.

Examples of cancers include, but are not limited to, acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, Burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous T cell lymphoma, embryonal tumors, endometrial cancer, ependymoblastoma, ependymoma, esophageal cancer, esthesioneuroblastoma, Ewing sarcoma family of tumors, extracranial germ cell tumor, extragonadal germ cell tumor, extrahepatic bile duct cancer, eye cancer fibrous histiocytoma of bone and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, Hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone, medulloblastoma, medulloepithelioma, melanoma, Merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), myeloid leukemia, acute myeloid leukemia (AML), multiple myeloma, myeloproliferative disorders, nasal cavity and paranasal sinus cancer, nasopharyngeal cancer, neuroblastoma, non-Hodgkin lymphoma, non-small cell lung cancer, oral cancer, oral cavity cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, Sezary syndrome, small cell lung cancer, small intestine cancer, soft tissue sarcoma, squamous cell carcinoma, squamous neck cancer, stomach (gastric) cancer, supratentorial primitive neuroectodermal tumors, T cell lymphoma, cutaneous cancer, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenstrom macroglobulinemia, and Wilms Tumor.

In some aspects, the cancer is a B cell malignancy. Examples of B cell malignancies include, but are not limited to, non-Hodgkin's lymphomas (NHL), diffuse large B cell lymphoma (DLBCL), small lymphocytic lymphoma (SLL/CLL), mantle cell lymphoma (MCL), follicular lymphoma (FL), marginal zone lymphoma (MZL), extranodal (e.g., MALT) lymphoma, nodal (e.g., monocytoid B cell) lymphoma, splenic lymphoma, diffuse large cell lymphoma, B cell chronic lymphocytic leukemia/lymphoma, Burkitt's lymphoma and lymphoblastic lymphoma.

A “cancer” may be associated with a surface antigen, such as a cancer/tumor antigen, or a cancer/tumor marker.

As used herein, the term “cancer antigen” is equivalent to “tumor antigen” and refers to an antigen that is differentially expressed by cancer cells and can therefore be exploited to target cancer cells. Cancer antigens are antigens that can potentially stimulate apparently tumor-specific immune responses. Some of these antigens are encoded, although not necessarily expressed, by normal cells; these antigens can be characterized as those that are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3 (defined by immunity), MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively.

Other cancer antigens include, but are not limited to, 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor-2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), κ-light chain, LAGE (L antigen), LDLR/FUT (low density lipid receptor/GDP-L-fucose: b-D-galactosidase 2-a-Lfucosyltransferase), LeY (Lewis-Y antibody), L1 CAM (L1 cell adhesion molecule), MAGE (melanoma antigen), MAGE-Al (melanoma-associated antigen 1), mesothelin, murine CMV infected cells, MART-1/Melan-A (melanoma antigen recognized by T cells- I/melanoma antigen A), MC1 R (melanocortin 1 receptor), yosin/m (myosin mutated), MUC1 (mucin 1), MUM-1, -2, -3 (melanoma ubiquitous mutated-1, -2, -3), NA88-A (NA cDNA clone of patient M88), NKG2D (natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190KD bcr-abl), Pml/RARa (promyelocytic leukaemia/retinoic acid receptor a), PRAME (preferentially expressed antigen of melanoma), PSA (prostate-specific antigen), PSCA (prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), synovial sarcoma X-1, -2, -3, -4 (SSX-1, -2, -3, -4), TAA (tumor-associated antigen), TAG-72 (tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), or WT1 (Wilms' tumor gene).

The T cell population of the present invention may be administered either alone or as a pharmaceutical composition. Preferably, the pharmaceutical composition comprises a pharmaceutically acceptable excipient.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene- polyoxypropylene- block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The T cell population of the present invention may be administered to a subject in any suitable manner, including by injection, ingestion, transfusion, implantation or transplantation. In some embodiments, T cell population may be administered to a subject by parenteral administration. In certain embodiments, the T cell populations described herein may be administered to a subject subcutaneously, intradermally, intratumorally, intranodally, intramedullary, intramuscularly, intrasternally, by intravenous (i.v.) injection, by infusion techniques or intraperitoneally. In particular embodiments, the T cell population of the present invention may be administered to a subject by intradermal or subcutaneous injection. In some embodiments, the T cell population of the present invention may be administered by i.v. injection. In some embodiments, the T cell population may be injected directly into a lymph node or a tumor site.

In some embodiments, the subject is administered (or is to be administered) with autologous cells. In some embodiments, the subject is administered (or is to be administered) with heterologous cells. In some embodiments, the subject is administered (or is to be administered) with allogenic cells.

The quantity and frequency of administration of the T cells will be determined by such factors as the condition of the subject and the type and severity of the subject's disease, although appropriate dosages may be determined by clinical trials.

When an “effective amount” or “therapeutic amount” is indicated, the precise amount of the T cell population or composition of the present invention to be administered may be determined with consideration of individual differences in age, weight, antibody titer, and condition of the subject. It can generally be stated that a pharmaceutical composition comprising the T cells as described herein may be administered at a dosage of at least 1×10², 1×10³, 1×10⁴, 1×10⁵, 1×10⁶, 1×10⁷, 1×10⁸ or 1×10⁹ cells/kg body weight or 1×10⁵ to 100×10⁵ cells/kg body weight, including all integer values within those ranges. T cell compositions may also be administered multiple times at any of these dosages or any combination thereof. T cells can be administered by using infusion techniques that are commonly known in immunotherapy. The optimal dosage and treatment regimen for a particular subject can readily be determined by monitoring the subject for signs of disease and adjusting the treatment accordingly.

In some embodiments, the subject (e.g., human) receives an initial administration of a

T cell or population of the invention, and one or more subsequent administrations, wherein the one or more subsequent administrations are administered less than 15 days, e.g., 14, 13, 12, 11, 10, 9, 8, 7, 6, 5, 4, 3, or 2 days after the previous administration.

In some embodiments, a therapeutically effective number of T cells of the invention is administered or is to be administered to the subject.

In some embodiments, the number of T cells of the T cell population of the invention administered to the subject is at least of 10², 10³, 10⁴, 10⁵, 10⁶, 10⁷, 10⁸ or 10⁹ cells.

In some embodiments, the number of T cells of the T cell population of the invention administered to the subject ranges from about 10² to about 10⁹, from about 10³ to about 10⁸, from about 10⁴ to about 10⁷, or from about 10⁵ to about 10⁶ cells.

In some embodiments, the number of T cells of the T cell population of the invention administered to the subject ranges from about 10² to about 10⁹, from about 10² to 10⁸, from about 10² to 10⁷, from about 10² to 10⁶, from about 10² to 10⁵, from about 10² to 10⁴, or from about 10² to 10³ cells. In some embodiments, the number of T cells of the T population of the invention administrated to the subject is about 10², about 10³, about 10⁴, about 10⁵, about 10⁶, about 10⁷, about 10⁸, or about 10⁹ cells.

In some embodiments, the number of T cells of the T cell population of the invention administered to the subject is at least 10², 10³, 10⁴, 10⁵, 10⁶, 10⁷, 10⁸ or 10⁹ cells/kg body weight.

In some embodiments, the number of T cells of the T cell population of the invention administered to the subject ranges from about 10² to 10⁹ cells/kg body weight or 10³ to 10⁸ cells/kg body weight, including all integer values within those ranges.

In some embodiments, the subject receives more than one administration of the T cell population of the invention per week, e.g., 2, 3, or 4 administrations of an T cell population of the invention administered per week to the subject.

In one embodiment, the T cell population of the present invention is to be administered before, concomitantly with or after another therapeutic drug.

In some embodiments, the T cells of the present invention may be administered to a subject in conjunction with (e.g., before, simultaneously or following) any number of relevant treatment modalities, including but not limited to treatment with agents such as e.g. chemotherapeutic agents.

Examples of chemotherapeutic agents include, without limitation, platinum coordination compounds (such as, e.g., cisplatin, carboplatin or oxalyplatin); taxane compounds (such as, e.g., paclitaxel or docetaxel); topoisomerase I inhibitors (such as, e.g., irinotecan or topotecan); topoisomerase II inhibitors (such as, e.g., etoposide or teniposide); vinca alkaloids (such as, e.g., vinblastine, vincristine or vinorelbine); anti-tumor nucleoside derivatives (such as, e.g., 5-fluorouracil, gemcitabine or capecitabine); alkylating agents (such as, e.g., nitrogen mustard or nitrosourea, cyclophosphamide, chlorambucil, carmustine or lomustine; anti-tumor anthracycline derivatives (suc has, e.g., daunorubicin, doxorubicin, idarubicin or mitoxantrone); anti-HER2 antibodies (such as, e.g., trastuzumab); estrogen receptor antagonists or selective estrogen receptor modulators (such as, e.g., tamoxifen, toremifene, droloxifene, faslodex or raloxifene);

aromatase inhibitors (such as, e.g., exemestane, anastrozole, letrazole or vorozole); differentiating agents (such as, e.g., retinoids, vitamin D and retinoic acid metabolism blocking agents [RAMBA] such as accutane); DNA methyl transferase inhibitors (such as, e.g., azacytidine); kinase inhibitors (such as, e.g., flavoperidol, imatinib mesylate or gefitinib); farnesyltransferase inhibitors; and HDAC inhibitors.

The T cells of the present invention may be administered to the subject before, after, or concomitant with the chemotherapeutic agent.

It is understood that the T cells, cell populations, and compositions described herein may be used in a method of treatment as described herein, may be for use as a medicament as described herein, may be for use in a treatment as described herein, and/or may be for use in the manufacture of a medicament for a treatment as described herein.

### TABLE OF SEQUENCES

Predicted signal peptide, when present, is indicated in bold letters in the following table.

| SEQ ID NO | Sequence |
|---|---|
| SEQ ID | MEPSSKKLTGRLMLAVGGAVLGSLQFGYNTGVINAPQKVIEEFYNQTWVHRYGESIL |
| NO: 1 | PTTLTTLWSLSVAIFSVGGMIGSFSVGLFVNRFGRRNSMLMMNLLAFVSAVLMGFSK |
| | LGKSFEMLILGRFIIGVYCGLTTGFVPMYVGEVSPTALRGALGTLHQLGIVVGILIAQV |
| | FGLDSIMGNKDLWPLLLSIIFIPALLQCIVLPFCPESPRFLLINRNEENRAKSVLKKLRGT |
| | ADVTHDLQEMKEESRQMMREKKVTILELFRSPAYRQPILIAVVLQLSQQLSGINAVFY |
| | YSTSIFEKAGVQQPVYATIGSGIVNTAFTVVSLFVVERAGRRTLHLIGLAGMAGCAIL |
| | MTIALALLEQLPWMSYLSIVAIFGFVAFFEVGPGPIPWFIVAELFSQGPRPAAIAVAGFS |
| | NWTSNFIVGMCFQYVEQLCGPYVFIIFTVLLVLFFIFTYFKVPETKGRTFDEIASGFRQG |
| | GASQSDKTPEELFHPLGADSQV |
| SEQ ID | VSKGEELFTGVVPILVELDGDVNGHKFSVSGEGEGDATYGKLTLKFICTTGKLPVPWP |
| NO: 2 | TLVTTLTYGVQCFSRYPDHMKQHDFFKSAMPEGYVQERTIFFKDDGNYKTRAEVKFE |
| | GDTLVNRIELKGIDFKEDGNILGHKLEYNYNSHNVYIMADKQKNGIKVNFKIRHNIED |
| | GSVQLADHYQQNTPIGDGPVLLPDNHYLSTQSALSKDPNEKRDHMVLLEFVTAAGIT |
| | LGMDELYK |
| SEQ ID | APSTCSKPTCPPPELLGGPSVFIFPPKPKDTLMISRTPEVTCVVVDVSQDDPEVQFTWYI |
| NO: 3 | NNEQVRTARPPLREQQFDCTIRVVSTLPIAHQDWLRGKEFKCKVHNKALPAPIEKTIS |
| | KARGQPLEPKVYTMGPPREELSSRSVSLTCMINGFYPSDISVEWEKNGKAEDNYKTTP |
| | AVLDSDGSYFLYSKLSVPTSEWQRGDVFTCSVMHEALHNHYTQKSISRSPGK |
| SEQ ID | VDVPRDCGCKPCICTVPEVSSVFIFPPKPKDVLTITLTPKVTCVVVDISKDDPEVQFSW |
| NO: 4 | FVDDVEVHTAQTQPREEQFNSTFRSVSELPIMHQDWLNGKEFKCRVNSAAFPAPIEKT |
| | ISKTKGRPKAPQVYTIPPPKEQMAKDKVSLTCMITDFFPEDITVEWQWNGQPAENYK |
| | NTQPIMDTDGSYFVYSKLNVQKSNWEAGNTFTCSVLHEGLHNHHTEKSLSHSPGK |
| SEQ ID | MGKFLATLILFFQFCPLILGDYSPSCCTLTIGVSSYHSKPCNPAQPVCSWTLDLLALS |
| NO: 5 | ADQALQPPCPNLVSYSSYHATYSLYLFPHWIKKPNRNGGGYYSASYSDPCSLKCPYL |
| | GCQSWTCPYTGAVSSPYWKFQQDVNFTQEVSRLNINLHFSKCGFPFSLLVDAPGYDPI |
| | WFLNTEPSQLPPTAPPLLPHSNLDHILEPSIPWKSKLLTLVQLTLQSTNYTCIVCIDRAS |
| | LSTWHVLYSPNVSVPSSSSTPLLYPSLALPAPHLTLPFNWTHCFDPQIQAIVSSPCHNSL |
| | ILPPFSLSPVPTLGSRSRR |
| SEQ ID | MGKFLATLILFFQFCPLIFGDYSPSCCTLTIGVSSYHSKPCNPAQPVCSWTLDLLALS |
| NO: 6 | ADQALQPPCPNLVSYSSYHATYSLYLFPHWTKKPNRNGGGYYSASYSDPCSLKCPYL |
| | GCQSWTCPYTGAVSSPYWKFQHDVNFTQEVSRLNINLHFSKCGFPFSLLVDAPGYDPI |
| | WFLNTEPSQLPPTAPPLLPHSNLDHILEPSIPWKSKLLTLVQLTLQSTNYTCIVC |
| SEQ ID | MGKFLATLILFFQFCPLIFGDYSPSCCTLTIGVSSYHSKPCNPAQPVCSWTLDLLALS |
| NO: 7 | ADQALQPPCPNLVSYSSYHATYSLYLFPHWTKKPNRNGGGYYSASYSDPCSLKCPYL |
| | GCQSWTCPYTGAVSSPYWKFQHDVNFTQEVSRLNINLHFSKCGFPFSLLVDAPGYDPI |
| | WFLNTEPSQ |
| SEQ ID | IKKPNPNGGGYYLASYSDPCSLKCPYLGCQSWTCPYTGAVSSPYWKFQQDV |
| NO: 8 | |
| SEQ ID | VKKPNRNGGGYYLASYSDPCSLKCPYLGCQSWTCPYTGAVSSPYWKFQQDV |
| NO: 9 | |
| SEQ ID | IKKPNRNGGGYYLASYSDPCSLKCPYLGCQSWTCPYTGAVSSPYWKFQQDV |
| NO: 10 | |
| SEQ ID | IKKPNRNGGGYYLASYSDPCSLKCPYLGCQSWTCPYTGPVSSPYWKFQQDV |
| NO: 11 | |
| SEQ ID | IKKPNRNGGGYHSASYSDPCSLKCPYLGCQSWTCPYAGAVSSPYWKFQQDVNFTQE |
| NO: 12 | V |
| SEQ ID | MGNVFFLLLFSLTHFPPVQQSRCTLTVGISSYHSSPCSPTQPVCTWNLDLNSLTTDQ |
| NO: 13 | RLHPPCPNLITYSGFHKTYSLYLFPHWIKKPNRQGLGYYSPSYNDPCSLQCPYLGCQS |
| | WTCPYTGPVSSPSWKFHSDVNFTQEVSQVSLRLHFSKCGSSMTLLVDAPGYDPLWFI |
| | TSEPTQPPPTPPPLVHDSDLEHVLTPSTSWTTKMLKFIQLTLQSTNYSCMVCVDRSSLS |
| | SWHVLYTPNISIPQQTSSRTILFPSLALPAPPFQPFPWTHCYQPRLQAITTDDCNNSIILP |
| | PFSLAPVPPPATRRRR |
| SEQ ID | MGNVFFLLLFSLTHFPLAQQSRCTLTVGISSYHSSPCSPTQPVCTWNLDLNSLTTDQ |
| NO: 14 | RLHPPCPNLITYSGFHKTYSLYLFPHWIKKPNRQGLGYYSPSYNDPCSLQCPYLGCQS |
| | WTCPYTGPVSSPSWKFHSDVNFTQEVSQVSLRLHFSKCGSSMTLLVDAPGYDPL WFI |
| | TSEPTQPPPTSPPLVHDSDLEHVLTPSTSWTTKILKFIQLTLQSTNYSCMVC |
| SEQ ID | MGNVFFLLLFSLTHFPLAQQSRCTLTVGISSYHSSPCSPTQPVCTWNLDLNSLTTDQ |
| NO: 15 | RLHPPCPNLITYSGFHKTYSLYLFPHWIKKPNRQGLGYYSPSYNDPCSLQCPYLGCQS |
| | WTCPYTGPVSSPSWKFHSDVNFTQEVSQVSLRLHFSKCGSSMTLLVDAPGYDPLWFI |
| | TSEPTQ |
| SEQ ID | IRKPNRQGLGYYSPSYNDPCSLQCPYLGSQSWTCPYTAPVSTPSWNFHSDV |
| NO: 16 | |
| SEQ ID | MGKSGLYFSLICFYTLFPSSFGNPSRCTLFIGASSYHSDPCGSNHPRCTWRLDLFSLT |
| NO: 17 | KDQSLSPPCPGLVTYSQYHKPYSLYVFPHWIAKPDRRGLGYYSASYSDPCAIQCPYLG |
| | CQSWTCPYTGPVSNPHWKYTSDLNFTQEVSSISLHLHFSKCGSSFSFLLDAPGYDPVW |
| | LLSSQATQIPPTPAPLIQDSDLQHILEPSIPWSSKILNLILLALKSTNYSCMVCVDRSSLS |
| | SWHVLYDPLKAPSSPDPQAQSILRPSLAIPASNITPPFPWTHCYRPPLQAISSENCNNSV |
| | ILPPFSLSPIPDVSRPRKRR |
| SEQ ID | MGKSGLYFSLICFYTLFPSSFGNPSRCTLFIGASSYHSDPCGSNHPRCTWRLDLFSLT |
| NO: 18 | KDQSLSPPCPGLVTYSQYHKPYSLYVFPHWIAKPDRRGLGYYSASYSDPCAIQCPYLG |
| | CQSWTCPYTGPVSNPHWKYTSDLNFTQEVSSISLHLHFSKCGSSFSFLLDAPGYDPVW |
| | LLSSQA |
| SEQ ID | MGNVLFLTLLATLGIPVLQASRCTITVGISSYHSSPCSPAQPLCTWALDLVSITKDQL |
| NO: 19 | LYPPCQNLITYSNYHKTYSLYLFPHWVQKPLRRGLGYYSASYSDPCSLQCPYLGSQS |
| | WTCPYTGPVSSPTWRFSTDVNFTQEVSRVSLKLHFSKCGSSLTLLIDAPGYDPLWYLT |
| | SEPTQEPPTPPPLVSDSDLEHVLTPSASWASKMLTLIHLTLQSTNYSCMVCIDRASLSS |
| | WHVLYTPNISSNAPSKPIVRPSLALSAPRPQPFPWTHCYQPQVQAVTTAKCNNSIILPP |
| | FSLSPLPGAPLTRRRR |
| SEQ ID | MGNVLFLTLLATLGIPVLQASRCTITVGISSYHSSPCSPAQPLCTWALDLVSITKDQL |
| NO: 20 | LYPPCQNLITYSNYHKTYSLYLFPHWVQKPLRRGLGYYSASYSDPCSLQCPYLGSQS |
| | WTCPYTGPVSSPTWRFSTDVNFTQEVSRVSLKLHFSKCGSSLTLLIDAPGYDPLWYLT |
| | SEPTQEPPTPPPLVSDSDLEHVLTPSASWASKMLTLIHLTLQSTNYSCMVC |
| SEQ ID | MGNVLFLTLLATLGIPVLQASRCTITVGISSYHSSPCSPAQPLCTWALDLVSITKDQL |
| NO: 21 | LYPPCQNLITYSNYHKTYSLYLFPHWVQKPLRRGLGYYSASYSDPCSLQCPYLGSQS |
| | WTCPYTGPVSSPTWRFSTDVNFTQEVSRVSLKLHFSKCGSSLTLLIDAPGYDPLWYLT |
| | SEPTQ |
| SEQ ID | MGKFLATLILFFQFCPLILGDYSPSCCTLTIGVSSYLSKPCNPAQPVCSWTLDLLALS |
| NO: 22 | ADQALQPPCPNLVSYSSYHATYSLYLFPHWIKKPNRNGGGYYSASYSDPCSLKCPYL |
| | GCQSWTCPYTGVVSSPYWKFQQDVNFTQEVSHLNINLHFSKCGFPFSLLIDAPGYDPI |
| | WFLNTEPSQLPPTAPPLLPHSNLDHILEPSIPWKSKLLTLVQLTLQSTNYTCIVCIDRAS |
| | LSTWHVLYSPNVSVPSSSSTPLLYPSLALPAPHLTLPFNWTHCFDPQIQAIVSSPCHNSL |
| | ILPPFSLSPVPTLGSRSRR |
| SEQ ID | MGKFLATLILFFQFCPLILGDYSPSCCILTIGVSSYHSKPCNPAQPVCSWTLDLLALS |
| NO: 23 | ADQALQPPCPNLVSYSSYHATYSLYLFPHWIKKPNRNGGGYYSASYSDPCSLKCPYL |
| | GCQSWTCPYTGAVSSPYWKFQQDVNFTQEVSHLNINLHFSKCGFPFSLLIDAPGYDPI |
| | WFLNTE |
| SEQ ID | MGKIIAFLLFHLTCITITKQSRCTLTVGVSSYHSSPCSLAQPICTWDLDLHSLTTDQR |
| NO: 24 | LYPPCPNLVSYSNFHKSYSLYLFPHWVKKPNRQGLGYYSASYSDPCSLQCPYLGSQS |
| | WTCPYTGPISSPSWRFHRDVNFTQEVNHVTLRLHFSRCGSSMTLLIDAPGYDPLWFISS |
| | EPTQPPPTSPPLVRDSDLEHILTPSSSWATRMLTLIQLTLQSTNYSCMVCIDRTSLSSWH |
| | VLYTPNISASPGGDSLPILYPSLALPAPQPQPFSWSHCYQPHLQAVTTANCNNSIVLPPF |
| | SLTPVPSPGTRSRR |
| SEQ ID | MGKIIAFLLFHLTCITITKQSRCTLTVGVSSYHSSPCSLAQPICTWDLDLHSLTTDQR |
| NO: 25 | LYPPCPNLVSYSNFHKSYSLYLFPHWVKKPNRQGLGYYSASYSDPCSLQCPYLGSQS |
| | WTCPYTGPISSPSWRFHRDVNFTQEVNHVTLRLHFSRCGSSMTLLIDAPGYDPLWFISS |
| | E |
| SEQ ID | MGKFGLYCLVHLYILLPASSGNPSRCTLFIGASSYHSSPCGSSLPRCTWNLDLFSLTK |
| NO: 26 | DQSLSPPCPDLITYSQYHKPYSLYVFPHWITKPNRRGLGYYSASYSDPCAIQCPYLGCQ |
| | SWTCPYTGPVSSPHWRYTYDLNFTQEVSSVSLHLHFSKCGSSFSFLLDAPGYDPVWFL |
| | SSQATQAPPTPAPLIRDSDLQYILEPPIPWSSKILNLILLTLKSTNYSCMVCVDRSSLSS |
| | WHVLYGPTQVPSPPDPQARSILRPALAIPASNITPPFPWTHCYRPPPQAISSENCNNSVV |
| | LPPFSLSPIPNVSRPRKRR |
| SEQ ID | MGKSGFYFCFIYTLFPASFGNPSRCTLFIGASSYHSDPCGSNHPQCTWRLDLFSLTRD |
| NO: 27 | QSLSPPCPDLVTYSQYHKPYSLYVFPHWMAKPNRQGLGYYSASYSDPCAIQCPYLGC |
| | QSWTCPYTGPVSSPHWKYSSDLNFTQEVSSISLHLHFSKCGSSFSFLLDAPGYDPVWF |
| | LSSQATQV |
| SEQ ID | MGKSLFFFCIIVQACLPTLCDRGPSCCTLTVGVSSYHSKPCNPTQPICSWTLDLLTLT |
| NO: 28 | TDQALQPPCPNLIGYSNYHATYPLYLFPHWVKKPNRGGGGYYSASYSDPCSLKCPYL |
| | GCQSWTCPYTGAVSGPYWKYQTDVNFTQDVSRLTVHLHFSKCGFPFSLLIDAPGYDP |
| | IWLINSEPSQLPPTAPPLLPHSNLDHILEPSIPWKSKLLTLVQLTLQSTNYTCIVCLDRAS |
| | LSTWHVLYSPNMSVPSSPSVPLIYPSLALPAPHLALPFNWTHCFDPKLQAITSAHCYN |
| | ALILPPFSLSPVPALSHRTRR |
| SEQ ID | MGNVFFLLLFSLTHFPLAQQSRCTLTVGISSYHSSPCSPTQPVCTWNLDLNSLTTDQ |
| NO: 29 | RLHPPCPNLITYSGFHKTYSLYLFPHWIKKPNRQGLGYYSPSYNDPCSLQCPYLGCQS |
| | WTCPYTGPVSSPSWKFHSDVNFTQEVSQVSLRLHFSKCGSSMTLLVDAPGYDPLWFI |
| | TSEPTQGSVSKGEELFTGVVPILVELDGDVNGHKFSVSGEGEGDATYGKLTLKFICTT |
| | GKLPVPWPTLVTTLTYGVQCFSRYPDHMKQHDFFKSAMPEGYVQERTIFFKDDGNY |
| | KTRAEVKFEGDTLVNRIELKGIDFKEDGNILGHKLEYNYNSHNVYIMADKQKNGIKV |
| | NFKIRHNIEDGSVQLADHYQQNTPIGDGPVLLPDNHYLSTQSALSKDPNEKRDHMVL |
| | LEFVTAAGITLGMDELYK |
| SEQ ID | MGNVFFLLLFSLTHFPLAQQSRCTLTVGISSYHSSPCSPTQPVCTWNLDLNSLTTDQ |
| NO: 30 | RLHPPCPNLITYSGFHKTYSLYLFPHWIKKPNRQGLGYYSPSYNDPCSLQCPYLGCQS |
| | WTCPYTGPVSSPSWKFHSDVNFTQEVSQVSLRLHFSKCGSSMTLLVDAPGYDPLWFI |
| | TSEPTQGSAPSTCSKPTCPPPELLGGPSVFIFPPKPKDTLMISRTPEVTCVVVDVSQDDP |
| | EVQFTWYINNEQVRTARPPLREQQFDCTIRVVSTLPIAHQDWLRGKEFKCKVHNKAL |
| | PAPIEKTISKARGQPLEPKVYTMGPPREELSSRSVSLTCMINGFYPSDISVEWEKNGKA |
| | EDNYKTTPAVLDSDGSYFLYSKLSVPTSEWQRGDVFTCSVMHEALHNHYTQKSISRS |
| | PGK |
| SEQ ID | MGNVFFLLLFSLTHFPLAQQSRCTLTVGISSYHSSPCSPTQPVCTWNLDLNSLTTDQ |
| NO: 31 | RLHPPCPNLITYSGFHKTYSLYLFPHWIKKPNRQGLGYYSPSYNDPCSLQCPYLGCQS |
| | WTCPYTGPVSSPSWKFHSDVNFTQEVSQVSLRLHFSKCGSSMTLLVDAPGYDPL WFI |
| | TSEPTQGSVDVPRDCGCKPCICTVPEVSSVFIFPPKPKDVLTITLTPKVTCVVVDISKDD |
| | PEVQFSWFVDDVEVHTAQTQPREEQFNSTFRSVSELPIMHQDWLNGKEFKCRVNSAA |
| | FPAPIEKTISKTKGRPKAPQVYTIPPPKEQMAKDKVSLTCMITDFFPEDITVEWQWNG |
| | QPAENYKNTQPIMDTDGSYFVYSKLNVQKSNWEAGNTFTCSVLHEGLHNHHTEKSL |
| | SHSPGK |
| SEQ ID | DYSPSCCTLTIGVSSYHSKPCNPAQPVCSWTLDLLALSADQALQPPCPNLVSYSSYHA |
| NO: 32 | TYSLYLFPHWIKKPNRNGGGYYSASYSDPCSLKCPYLGCQSWTCPYTGAVSSPYWKF |
| | QQDVNFTQEVSRLNINLHFSKCGFPFSLLVDAPGYDPIWFLNTEPSQLPPTAPPLLPHS |
| | NLDHILEPSIPWKSKLLTLVQLTLQSTNYTCIVCIDRASLSTWHVLYSPNVSVPSSSSTP |
| | LLYPSLALPAPHLTLPFNWTHCFDPQIQAIVSSPCHNSLILPPFSLSPVPTLGSRSRR |
| SEQ ID | DYSPSCCTLTIGVSSYHSKPCNPAQPVCSWTLDLLALSADQALQPPCPNLVSYSSYHA |
| NO: 33 | TYSLYLFPHWTKKPNRNGGGYYSASYSDPCSLKCPYLGCQSWTCPYTGAVSSPYWK |
| | FQHDVNFTQEVSRLNINLHFSKCGFPFSLLVDAPGYDPIWFLNTEPSQLPPTAPPLLPHS |
| | NLDHILEPSIPWKSKLLTLVQLTLQSTNYTCIVC |
| SEQ ID | DYSPSCCTLTIGVSSYHSKPCNPAQPVCSWTLDLLALSADQALQPPCPNLVSYSSYHA |
| NO: 34 | TYSLYLFPHWTKKPNRNGGGYYSASYSDPCSLKCPYLGCQSWTCPYTGAVSSPYWK |
| | FQHDVNFTQEVSRLNINLHFSKCGFPFSLLVDAPGYDPIWFLNTEPSQ |
| SEQ ID | SRCTLTVGISSYHSSPCSPTQPVCTWNLDLNSLTTDQRLHPPCPNLITYSGFHKTYSLY |
| NO: 35 | LFPHWIKKPNRQGLGYYSPSYNDPCSLQCPYLGCQSWTCPYTGPVSSPSWKFHSDVN |
| | FTQEVSQVSLRLHFSKCGSSMTLLVDAPGYDPLWFITSEPTQPPPTPPPLVHDSDLEHV |
| | LTPSTSWTTKMLKFIQLTLQSTNYSCMVCVDRSSLSSWHVLYTPNISIPQQTSSRTILFP |
| | SLALPAPPFQPFPWTHCYQPRLQAITTDDCNNSIILPPFSLAPVPPPATRRRR |
| SEQ ID | SRCTLTVGISSYHSSPCSPTQPVCTWNLDLNSLTTDQRLHPPCPNLITYSGFHKTYSLY |
| NO: 36 | LFPHWIKKPNRQGLGYYSPSYNDPCSLQCPYLGCQSWTCPYTGPVSSPSWKFHSDVN |
| | FTQEVSQVSLRLHFSKCGSSMTLLVDAPGYDPLWFITSEPTQPPPTSPPLVHDSDLEHV |
| | LTPSTSWTTKILKFIQLTLQSTNYSCMVC |
| SEQ ID | SRCTLTVGISSYHSSPCSPTQPVCTWNLDLNSLTTDQRLHPPCPNLITYSGFHKTYSLY |
| NO: 37 | LFPHWIKKPNRQGLGYYSPSYNDPCSLQCPYLGCQSWTCPYTGPVSSPSWKFHSDVN |
| | FTQEVSQVSLRLHFSKCGSSMTLLVDAPGYDPLWFITSEPTQ |
| SEQ ID | NPSRCTLFIGASSYHSDPCGSNHPRCTWRLDLFSLTKDQSLSPPCPGLVTYSQYHKPYS |
| NO: 38 | LYVFPHWIAKPDRRGLGYYSASYSDPCAIQCPYLGCQSWTCPYTGPVSNPHWKYTSD |
| | LNFTQEVSSISLHLHFSKCGSSFSFLLDAPGYDPVWLLSSQATQIPPTPAPLIQDSDLQHI |
| | LEPSIPWSSKILNLILLALKSTNYSCMVCVDRSSLSSWHVLYDPLKAPSSPDPQAQSILR |
| | PSLAIPASNITPPFPWTHCYRPPLQAISSENCNNSVILPPFSLSPIPDVSRPRKRR |
| SEQ ID | NPSRCTLFIGASSYHSDPCGSNHPRCTWRLDLFSLTKDQSLSPPCPGLVTYSQYHKPYS |
| NO: 39 | LYVFPHWIAKPDRRGLGYYSASYSDPCAIQCPYLGCQSWTCPYTGPVSNPHWKYTSD |
| | LNFTQEVSSISLHLHFSKCGSSFSFLLDAPGYDPVWLLSSQA |
| SEQ ID | SRCTITVGISSYHSSPCSPAQPLCTWALDLVSITKDQLLYPPCQNLITYSNYHKTYSLYL |
| NO: 40 | FPHWVQKPLRRGLGYYSASYSDPCSLQCPYLGSQSWTCPYTGPVSSPTWRFSTDVNF |
| | TQEVSRVSLKLHFSKCGSSLTLLIDAPGYDPLWYLTSEPTQEPPTPPPLVSDSDLEHVL |
| | TPSASWASKMLTLIHLTLQSTNYSCMVCIDRASLSSWHVLYTPNISSNAPSKPIVRPSL |
| | ALSAPRPQPFPWTHCYQPQVQAVTTAKCNNSIILPPFSLSPLPGAPLTRRRR |
| SEQ ID | SRCTITVGISSYHSSPCSPAQPLCTWALDLVSITKDQLLYPPCQNLITYSNYHKTYSLYL |
| NO: 41 | FPHWVQKPLRRGLGYYSASYSDPCSLQCPYLGSQSWTCPYTGPVSSPTWRFSTDVNF |
| | TQEVSRVSLKLHFSKCGSSLTLLIDAPGYDPLWYLTSEPTQEPPTPPPLVSDSDLEHVL |
| | TPSASWASKMLTLIHLTLQSTNYSCMVC |
| SEQ ID | SRCTITVGISSYHSSPCSPAQPLCTWALDLVSITKDQLLYPPCQNLITYSNYHKTYSLYL |
| NO: 42 | FPHWVQKPLRRGLGYYSASYSDPCSLQCPYLGSQSWTCPYTGPVSSPTWRFSTDVNF |
| | TQEVSRVSLKLHFSKCGSSLTLLIDAPGYDPLWYLTSEPTQ |
| SEQ ID | DYSPSCCTLTIGVSSYLSKPCNPAQPVCSWTLDLLALSADQALQPPCPNLVSYSSYHA |
| NO: 43 | TYSLYLFPHWIKKPNRNGGGYYSASYSDPCSLKCPYLGCQSWTCPYTGVVSSPYWKF |
| | QQDVNFTQEVSHLNINLHFSKCGFPFSLLIDAPGYDPIWFLNTEPSQLPPTAPPLLPHSN |
| | LDHILEPSIPWKSKLLTLVQLTLQSTNYTCIVCIDRASLSTWHVLYSPNVSVPSSSSTPL |
| | LYPSLALPAPHLTLPFNWTHCFDPQIQAIVSSPCHNSLILPPFSLSPVPTLGSRSRR |
| SEQ ID | DYSPSCCILTIGVSSYHSKPCNPAQPVCSWTLDLLALSADQALQPPCPNLVSYSSYHAT |
| NO: 44 | YSLYLFPHWIKKPNRNGGGYYSASYSDPCSLKCPYLGCQSWTCPYTGAVSSPYWKFQ |
| | QDVNFTQEVSHLNINLHFSKCGFPFSLLIDAPGYDPIWFLNTE |
| SEQ ID | SRCTLTVGVSSYHSSPCSLAQPICTWDLDLHSLTTDQRLYPPCPNLVSYSNFHKSYSLY |
| NO: 45 | LFPHWVKKPNRQGLGYYSASYSDPCSLQCPYLGSQSWTCPYTGPISSPSWRFHRDVN |
| | FTQEVNHVTLRLHFSRCGSSMTLLIDAPGYDPLWFISSEPTQPPPTSPPLVRDSDLEHIL |
| | TPSSSWATRMLTLIQLTLQSTNYSCMVCIDRTSLSSWHVLYTPNISASPGGDSLPILYPS |
| | LALPAPQPQPFSWSHCYQPHLQAVTTANCNNSIVLPPFSLTPVPSPGTRSRR |
| SEQ ID | SRCTLTVGVSSYHSSPCSLAQPICTWDLDLHSLTTDQRLYPPCPNLVSYSNFHKSYSLY |
| NO: 46 | LFPHWVKKPNRQGLGYYSASYSDPCSLQCPYLGSQSWTCPYTGPISSPSWRFHRDVN |
| | FTQEVNHVTLRLHFSRCGSSMTLLIDAPGYDPLWFISSE |
| SEQ ID | NPSRCTLFIGASSYHSSPCGSSLPRCTWNLDLFSLTKDQSLSPPCPDLITYSQYHKPYSL |
| NO: 47 | YVFPHWITKPNRRGLGYYSASYSDPCAIQCPYLGCQSWTCPYTGPVSSPHWRYTYDL |
| | NFTQEVSSVSLHLHFSKCGSSFSFLLDAPGYDPVWFLSSQATQAPPTPAPLIRDSDLQY |
| | ILEPPIPWSSKILNLILLTLKSTNYSCMVCVDRSSLSSWHVLYGPTQVPSPPDPQARSIL |
| | RPALAIPASNITPPFPWTHCYRPPPQAISSENCNNSVVLPPFSLSPIPNVSRPRKRR |
| SEQ ID | NPSRCTLFIGASSYHSDPCGSNHPQCTWRLDLFSLTRDQSLSPPCPDLVTYSQYHKPYS |
| NO: 48 | LYVFPHWMAKPNRQGLGYYSASYSDPCAIQCPYLGCQSWTCPYTGPVSSPHWKYSS |
| | DLNFTQEVSSISLHLHFSKCGSSFSFLLDAPGYDPVWFLSSQATQV |
| SEQ ID | CDRGPSCCTLTVGVSSYHSKPCNPTQPICSWTLDLLTLTTDQALQPPCPNLIGYSNYH |
| NO: 49 | ATYPLYLFPHWVKKPNRGGGGYYSASYSDPCSLKCPYLGCQSWTCPYTGAVSGPYW |
| | KYQTDVNFTQDVSRLTVHLHFSKCGFPFSLLIDAPGYDPIWLINSEPSQLPPTAPPLLPH |
| | SNLDHILEPSIPWKSKLLTLVQLTLQSTNYTCIVCLDRASLSTWHVLYSPNMSVPSSPS |
| | VPLIYPSLALPAPHLALPFNWTHCFDPKLQAITSAHCYNALILPPFSLSPVPALSHRTRR |
| SEQ ID | SRCTLTVGISSYHSSPCSPTQPVCTWNLDLNSLTTDQRLHPPCPNLITYSGFHKTYSLY |
| NO: 50 | LFPHWIKKPNRQGLGYYSPSYNDPCSLQCPYLGCQSWTCPYTGPVSSPSWKFHSDVN |
| | FTQEVSQVSLRLHFSKCGSSMTLLVDAPGYDPLWFITSEPTQGSVSKGEELFTGVVPIL |
| | VELDGDVNGHKFSVSGEGEGDATYGKLTLKFICTTGKLPVPWPTLVTTLTYGVQCFS |
| | RYPDHMKQHDFFKSAMPEGYVQERTIFFKDDGNYKTRAEVKFEGDTLVNRIELKGID |
| | FKEDGNILGHKLEYNYNSHNVYIMADKQKNGIKVNFKIRHNIEDGSVQLADHYQQNT |
| | PIGDGPVLLPDNHYLSTQSALSKDPNEKRDHMVLLEFVTAAGITLGMDELYK |
| SEQ ID | SRCTLTVGISSYHSSPCSPTQPVCTWNLDLNSLTTDQRLHPPCPNLITYSGFHKTYSLY |
| NO: 51 | LFPHWIKKPNRQGLGYYSPSYNDPCSLQCPYLGCQSWTCPYTGPVSSPSWKFHSDVN |
| | FTQEVSQVSLRLHFSKCGSSMTLLVDAPGYDPLWFITSEPTQGSAPSTCSKPTCPPPEL |
| | LGGPSVFIFPPKPKDTLMISRTPEVTCVVVDVSQDDPEVQFTWYINNEQVRTARPPLRE |
| | QQFDCTIRVVSTLPIAHQDWLRGKEFKCKVHNKALPAPIEKTISKARGQPLEPKVYTM |
| | GPPREELSSRSVSLTCMINGFYPSDISVEWEKNGKAEDNYKTTPAVLDSDGSYFLYSK |
| | LSVPTSEWQRGDVFTCSVMHEALHNHYTQKSISRSPGK |
| SEQ ID | SRCTLTVGISSYHSSPCSPTQPVCTWNLDLNSLTTDQRLHPPCPNLITYSGFHKTYSLY |
| NO: 52 | LFPHWIKKPNRQGLGYYSPSYNDPCSLQCPYLGCQSWTCPYTGPVSSPSWKFHSDVN |
| | FTQEVSQVSLRLHFSKCGSSMTLLVDAPGYDPLWFITSEPTQGSVDVPRDCGCKPCIC |
| | TVPEVSSVFIFPPKPKDVLTITLTPKVTCVVVDISKDDPEVQFSWFVDDVEVHTAQTQP |
| | REEQFNSTFRSVSELPIMHQDWLNGKEFKCRVNSAAFPAPIEKTISKTKGRPKAPQVY |
| | TIPPPKEQMAKDKVSLTCMITDFFPEDITVEWQWNGQPAENYKNTQPIMDTDGSYFV |
| | YSKLNVQKSNWEAGNTFTCSVLHEGLHNHHTEKSLSHSPGK |

### BRIEF DESCRIPTION OF THE DRAWINGS

FIG. 1: Schematic of experimental system used for the generation of T cell populations discriminated on the basis of GLUT1 expression levels.

FIG. 2: Evaluation of transduction efficacy of CD19-CD28 CAR (A) and CD19-4-1-BB CAR (B) constructs at day 7 in the GLUT1-Lo and GLUT1-Hi subsets of the CD4³⁰ or CD8+T cell populations, monitored by flow cytometry by protein L staining. Representative data from one of four independent experiments are shown (n=4 independent donors).

FIGS. 3: Evaluation by flow cytometry of the CD4 and CD8 expression profiles of cells transduced with the CD19-CD28 CAR or the CD19-4-1-BB CAR construct, at day 7 of expansion. Percentages of CD4³⁰ T cells and CD8⁺T cells in the GLUT1-Lo and GLUT1-Hi subsets are shown. Data are representative of the results in 1 of 4 independent experiments.

FIG. 4: Percentages of naïve (T_{NAIVE}, CD62L+/CD45RA+), central memory (Tcm, CD62L+/CD45RA-), effector memory (TEM, CD62L-/CD45RA-) and effector memory reexpressing CD45RA (T_{EMRA}, CD62L−/CD45RA+) among the CD4³⁰ T cells (A) and the CD8⁺T cells (B) within the GLUT1-Lo and GLUT1-Hi subsets at day 6 of culture. Expression data for 3 independent donors are shown.

FIG. 5: Tumor growth follow-up by bioluminescent imaging in NSG mice injected with CD19⁺GL-Nalm6 leukemia cells and adoptively transferred at day 3 with mock T cells, total CAR T cells, GLUT1-Lo CAR T cells or GLUT1-Hi CAR T cells.

FIG. 6: Tumor growth follow-up by bioluminescent imaging in NSG mice injected with CD19⁺GL-Nalm6 leukemia cells and adoptively transferred at day 3 with CD19-CD28 CAR T cells (A) or CD19-4-1BB CAR T cells (B). CAR T cells were either total or sorted GLUT1-Lo or GLUT1-Hi T cells as indicated.

FIG. 7: Percentage of human CD3⁺CAR T cells in the spleen of NSG mice injected with CD19-CAR-BB GLUT1-Lo T cells (A) or CD19-CAR-BB GLUT1-Hi T cells (B), measured by flow cytometry at day 43.

### EXAMPLES

The present invention is further illustrated by the following examples.

In order to test the respective anti-cancer activities of GLUT1-Hi T cells versus GLUT1-Lo T cells in leukemic tumor-bearing NSG mice, T cells were transduced with different CAR constructs and sorted on the basis of GLUT1 expression, before injection to the tumor-bearing mice (see the following examples).

Example 1: Experimental procedure for the generation of T cell subsets with low or high GLUT1 expression levels.

Material and Methods

### Healthy Donor Lymphocytes

Human healthy donor peripheral blood mononuclear cells were obtained from the NIH Blood Bank and were frozen in 10% FBS and stored in liquid nitrogen.

### Generation of Human CD19 CAR T Cells

Lentiviral vectors encoding CD19 CAR co-expressing either the CD28 or the 4-1BB co-stimulatory domain were produced by transient transfection of 293T cells (Lenti-X, Takara Bio). 293T cells were transfected using Lipofectamine 3000 (Life Technologies) with plasmids encoding packing and envelope vectors (pMDLg/pRRE, pMD.2G, pRS V-Rev, p3000), as well as a plasmid encoding the CD19 CAR under the control of the EFla promoter. Viral supernatant was harvested from transfected cells at 24 and 48 hours after transfection, and supernatants were centrifuged for 10 minutes at 3,000 RPM to remove cell debris.

Human lymphocytes were thawed and cultured at a concentration of 1×10⁶/mL. They were activated in AIM-V media at a 1:1 ratio of Dynabeads® Human T-Expander CD3/CD28 (Invitrogen) per cell in the presence of IL-2 (40 IU/mL) for 48 hours. T cells were then resuspended at 2×10⁶/mL in lentiviral supernatant (10 mls), supplemented with 5 mL AIM-V, 100 IU/mL IL-2, and 10 μg/mL protamine sulfate, and subsequently centrifuged at 2,000×g for 2 hours at 32° C. in 6-well plates. Plates were then incubated overnight at 37° C., and the process was repeated on day 3. After the second overnight incubation, CD3/CD28 microbeads were removed, T cells were sorted (see below) and resuspended at 0.3×10⁶/mL in AIM-V media supplemented with 100 IU/mL IL-2. They were then cultured for an additional 48-72 hours before use in experiments. After generation, T cells (both CAR-transduced and mock) were cultured in AIM-V medium supplemented with 5% heat-inactivated FBS, 100 U/mL penicillin, 100 U/mL streptomycin, 2 mmol/L 1-glutamine, 10 mmol/L HEPES, and 100 IU/mL IL-2.

### Cell Sorting Based on GLUT1 Expression

Surface GLUT1 expression was monitored by binding to the HTLV-receptor binding domain (RBD) ligand (SEQ ID NO: 15) fused to eGFP (fusion protein of sequence SEQ ID NO: 29) as previously described (Manel et al., 2003. Cell. 115(4):449-59; Kim et al., 2004. Retrovirology. 1:41) (Metafora Biosystems). For sorting of cells based on GLUT1 expression, 50×10⁶ cells were stained at day 4 post stimulation, i.e. immediately following the second transduction with the CD19-CAR, or at day 2 post stimulation, i.e. before transduction with the CD19-CAR (similar results, data not shown). Negative staining was evaluated in comparison with unstained cells. Cells designated as GLUT1-Lo and GLUT1-Hi were distinguished within the positively-stained population (generally >85% of all T cells) and irrespective of CD19-CAR staining. Within the positive population, GLUT1-Lo and GLUT1-Hi cells were identified as those staining with the 15% lowest and highest GLUT1 staining, respectively. Cells were sorted on a FACS Aria (BD Biosciences) and cultured for another 72-96 hours before evaluation.

### Flow Cytometry Analyses

The following human monoclonal antibodies were used for detection of cell surface proteins: CD3-APC/Cy7, CD8-BV605, CD4-APC-R700, CD4-Pacific Blue, and CD69-APC (all from BD Bioscience). Dead cells were identified using eFluor 506 fixable viability dye (Thermo Fisher Scientific). GFP-expressing leukemia was identified through the FITC channel.

Cell surface CAR expression was evaluated at day 7 either by staining with a conjugated CD19-Fc-PE protein or by staining with protein L (ThermoFisher) followed by incubation with streptavidin-PE. Staining was evaluated by flow cytometry performed on an LSR II Fortessa Flow Cytometer (BD Biosciences) and data were analyzed using FlowJo software.

### Mice

NOD-SCID-gc-l- (NSG) mice on a B16 background were provided by the National Cancer Institute from Frederick. The mice were housed in conventional, pathogen-free facilities at the National Cancer Institute at Bethesda Animal care and experiments were performed in accordance with the National Institutes of Health (NIH) guidelines.

### Cell Line

B-ALL cell lines included the GFP-luciferase-stably transduced Nalm6 line (Pediatric Oncology Branch, NCI, NIH, Bethesda, MD). Nalm6 was cultured in RPMI medium supplemented with 10% heat-inactivated FBS.

### In Vivo Tumor Experiments

Animal experiments were carried out under protocols approved by the NCI Bethesda Animal Care and Use Committee. B-ALL cell lines were IV injected into NSG mice (1×10⁶) and four days later, mice were intravenously injected with 2 million CAR T cells or an equivalent number of untransduced MOCK control T cells. Leukemia burden was evaluated using the Xenogen IVIS Lumina system (Caliper Life Sciences). Following intraperitoneal injection of mice with 3 mg D-luciferin (Caliper Life Sciences), mice were imaged 4 minutes later with an exposure time of 30 sec. Luminescence images were evaluated using Living Image Version 4.1 software (Caliper Life Sciences) as photons/s/cm2/ sr. Mice were harvested following expansion of leukemic cells in order to harvest spleens and bone marrow for phenotyping.

### Results

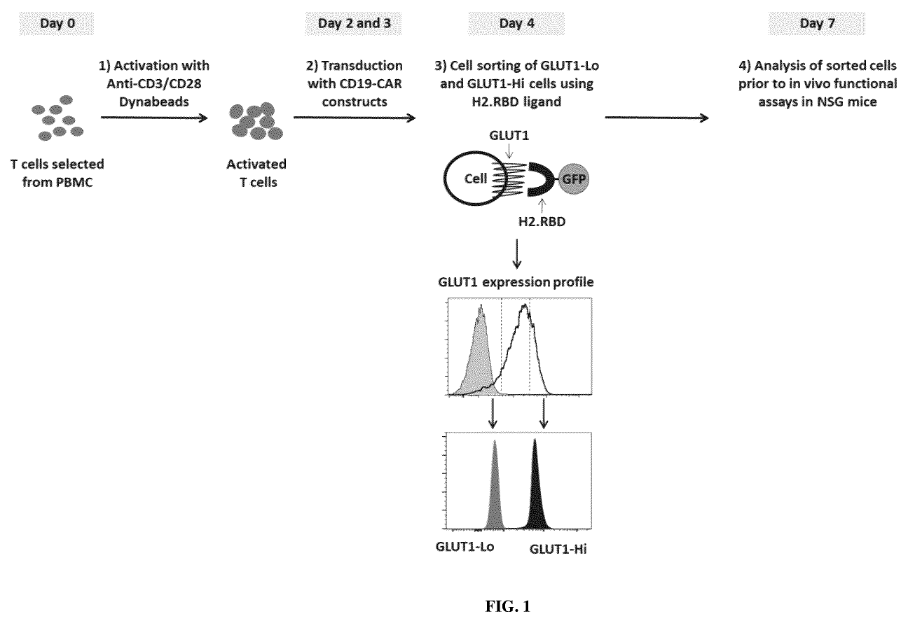

T cells were selected on the basis of GLUT1 glucose transporter expression (FIG. 1).

Example 2: GLUT1-Hi CD4³⁰ and CD8+T cells displayed higher transduction efficacy than GLUT1-Lo CD4³⁰ and CD8+T cells.

### Materials and Methods

T cells harboring the CD19-CD28 or CD19-4-1BB CAR constructs were generated, as described in Example 1. GLUT1-Lo and GLUT1-Hi cells were sorted at day 4, as described in Example 1. At day 7, transduction efficacy was evaluated by flow cytometry on gated CD4³⁰ and CD8+cells as a function of surface CD19-CAR expression levels, monitored by protein L staining, as described in Example 1.

### Results

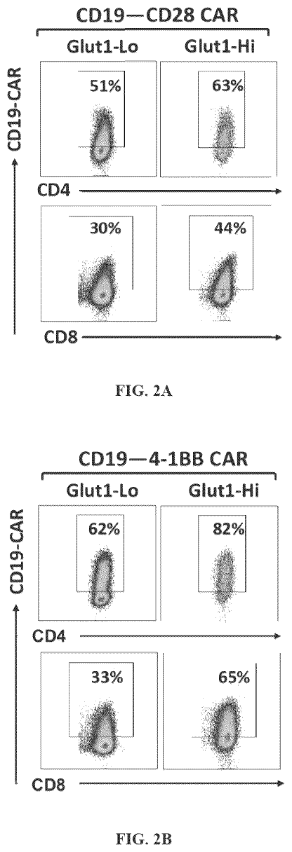

While both CD4+ and CD8+T cell subsets were transduced, there was a significantly higher transduction of CD4+ cells than CD8+ cells and, in both cases, GLUT1-Hi lymphocytes were transduced to a higher frequency than GLUT1-Lo lymphocytes (FIG. 2).

Example 3: GLUT1-Hi T Cells Exhibited a Skewed CD4/CD8 Ratio.

Materials and Methods

T cells harboring the CD19-CD28 or CD19-4-1BB CAR constructs were generated, as described in Example 1. GLUT1-Lo and GLUT1-Hi cells were sorted at day 4, as described in Example 1. At day 7, the CD4 and CD8 expression profiles of transduced cells were evaluated by flow cytometry.

### Results

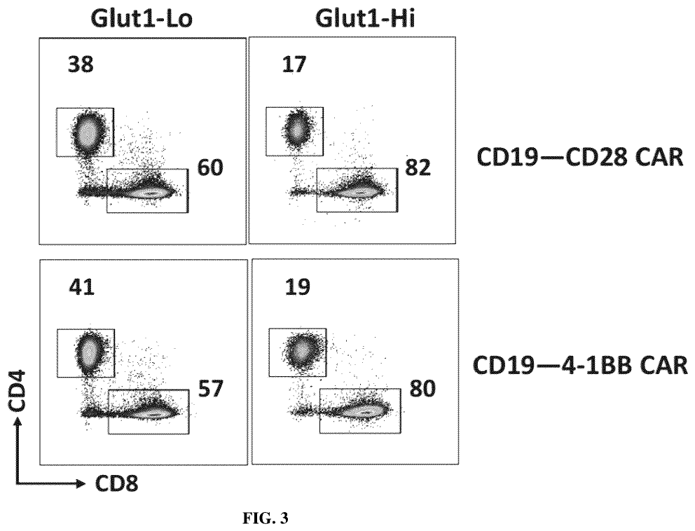

While GLUT1-Hi T cells were transduced to higher levels than GLUT1-Lo T cells, both subsets were transduced at significant levels. Interestingly, within the GLUT1-Hi subset, there was a skewed CD4+/CD8+T cell ratio of approximately 1:3, while this ratio was closer to 1:1 within the GLUT1-Lo subset (FIG. 3).

Example 4: The GLUT1-Hi subset comprised decreased CD8 naive T cells compared to the GLUT1-Lo subset.

### Materials and Methods

T cells harboring the CD19-CD28 or CD19-4-1BB CAR constructs were generated, as described in Example 1. GLUT1-Lo and GLUT1-Hi cells were sorted at day 4, as described in Example 1. At day 6, the phenotype of T lymphocytes within the GLUT1-Lo and GLUT1-Hi subsets was evaluated by flow cytometry by staining with CD62L and CD45RA markers, separating naïve (CD62L+/CD45RA+), central memory (TCM, CD62L+/CD45RA−), effector memory (EM, CD62L−/CD45RA−) and effector memory re-expressing CD45RA (TEMRA, CD62L−/CD45RA+).

### Results

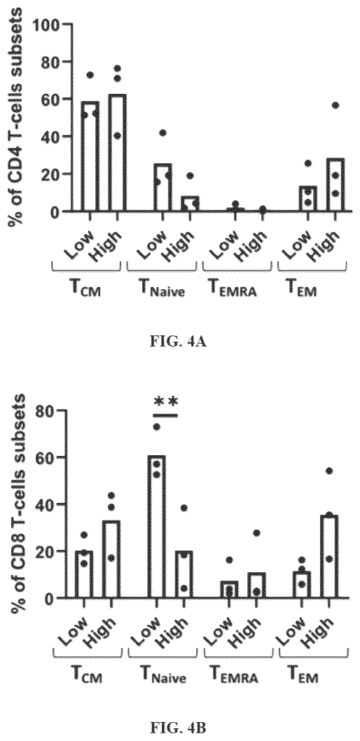

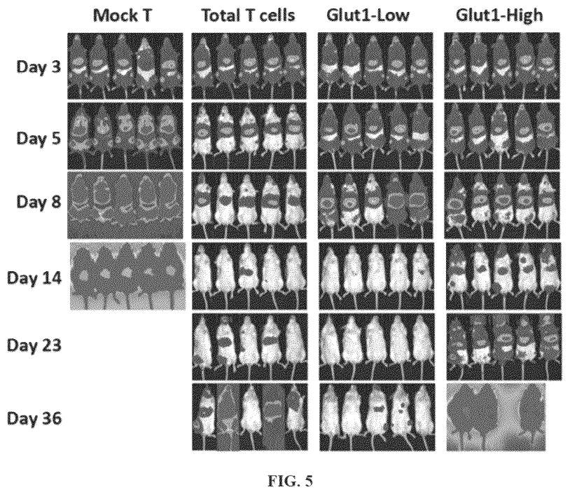

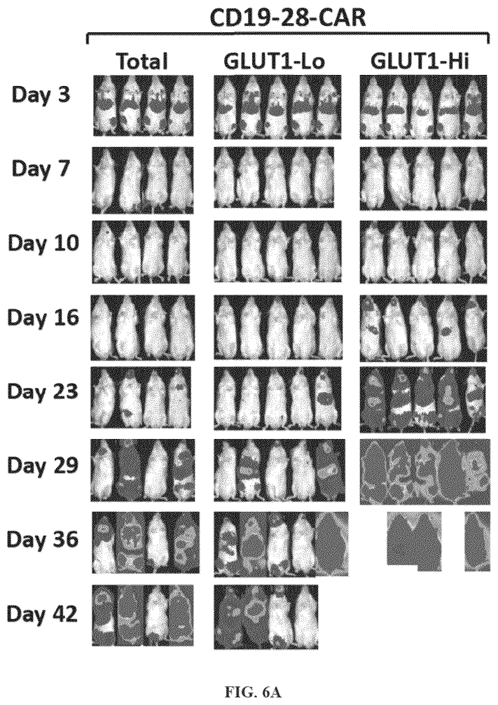

Differences in CD8+T cell phenotype were more marked than in CD4+T cells, with significantly higher percentages of naïve CD8+T cells in the GLUT1-Lo subset and trends towards higher effector memory and central memory CD8 lymphocytes in the GLUT1-Hi subset (FIG. 4).

Example 5: Potent anti-leukemic activity of T cells selected on the basis of GLUT1 expression levels.

### Materials and Methods

NSG mice were injected with 1×10⁶ CD19+GL-Nalm6 leukemia cells and at day 3, mice were adoptively transferred with 2×10⁶ CD19-BB-CAR T cells, either total or FACS-sorted on the basis of low or high GLUT1 expression, as described in Example 1. Tumor growth was followed by bioluminescent imaging, as described in Example 1.

### Results

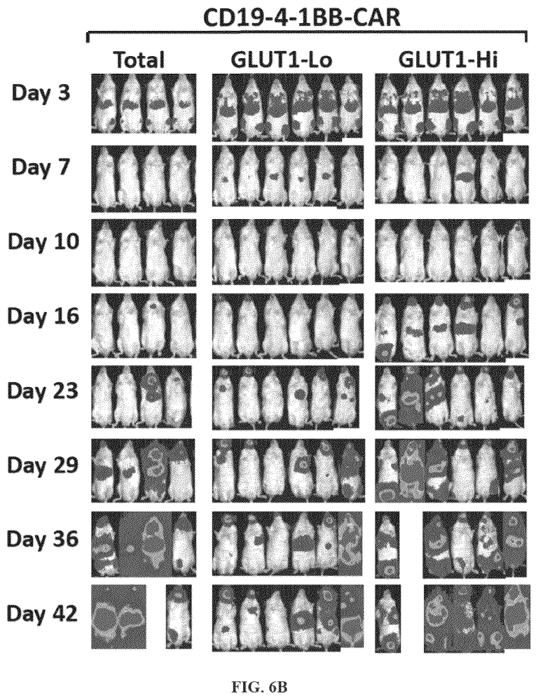

The activity of transferred GLUT1-Lo and GLUT1-Hi T cells was monitored in tumor-bearing NSG mice. Data from NSG mice harboring luciferase-positive CD19+Nalm6 leukemic cells demonstrated a substantially improved tumor rejection upon adoptive transfer of GLUT1-Lo as compared to GLUT1-Hi CD19-BB-CAR-T cells (FIG. 5).

Example 6: Potent anti-leukemic activity of T cells of various type selected on the basis of GLUT1 expression levels.

### Materials and Methods

NSG mice were injected with 1×10⁶ CD19+GL-Nalm6 leukemia cells and, at day 3, mice were adoptively transferred with 2×10⁶ CD19-28-CAR (FIG. 6A) or CD19-1-4BB-CAR (FIG. 6B) T cells, either total or FACS-sorted on the basis of low or high GLUT1 expression, as described in Example 1. Tumor growth was followed by bioluminescent imaging, as described in Example 1.

### Results

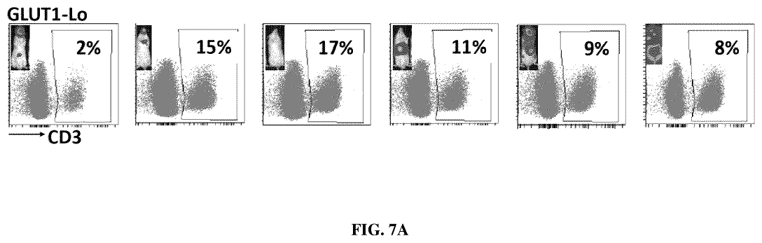

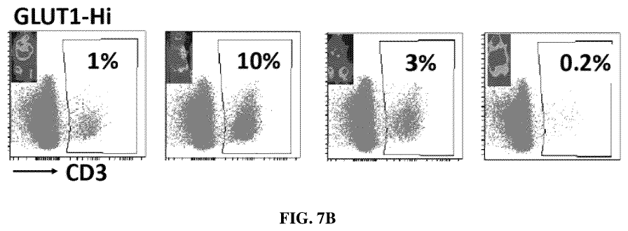

These experiments were repeated with multiple T cell donors and experiments were performed with both CD19-28-CAR and CD19-4-1BB-CAR T cells. As shown in FIG. 7, in a stress model using minimal numbers of adoptively transferred T cells, all mice receiving GLUT1-Hi T cells had significant tumor burden at day 43, with mice receiving the CD19-28-CAR construct having died from their leukemia. While 3 of 4 mice receiving total “conventional” CD19-28-CAR or CD19-4-1BB CAR T cells had significant leukemia burden or died from disease, 2 of 4 mice in the CD19-28-CAR GLUT1-Lo group and 5 of 6 mice in the CD19-4-1BB-CAR GLUT1-Lo group had low tumor burden (FIG. 6).

Example 7: Increased persistence of adoptively transferred T cells selected on the basis of low GLUT1 expression levels.

### Materials and Methods

NSG mice were injected with 1×10⁶ CD19+GL-Nalm6 leukemia cells and, at day 3, mice were adoptively transferred with 2×10⁶ FACS-sorted CD19-4-1BB-CAR GLUT1-Lo or GLUT1-Hi T cells, as described in Example 1. Tumor growth was followed by bioluminescent imaging, as described in Example 1. At day 43, percentage of human CD3+T cells in the spleen of each mouse was measured by flow cytometry to quantify persistence of adoptively transferred T cells.

### Results

Low tumor burden was associated with a higher percentage of adoptively transferred T cells in the GLUT1-Lo group than in the GLUT1-Hi group at day 43 post adoptive transfer (FIG. 7).

In conclusion, GLUT1-Lo T cells adoptively transferred to tumor-bearing mice are capable of better decreasing tumor burden than GLUT1-Hi T cells and total T cells. Therefore, T cells selected on the basis of low GLUT1 expression levels exhibits increased anti-cancer activity and effector function.

1-15. (canceled)

16. A method of selecting T cells with improved anti-cancer activity, said method comprising:
a) quantifying glucose transporter 1 (GLUT1) expression level at the cell surface of a population of T cells by using a GLUT1 ligand,
b) selecting T cells having a low GLUT1 expression level,
wherein said T cells having a low GLUT1 expression level have improved anti-cancer activity.

17. The method according to claim 16, comprising:
a0) contacting a population of T cells expressing GLUT1 at their cell surface, or susceptible to express GLUT1 at their cell surface, with a GLUT1 ligand,
a1) detecting and/or quantifying the binding of said GLUT1 ligand to GLUT1 at the cell surface of the T cells,
a2) quantifying GLUT1 expression level at the cell surface of the T cells,
b) selecting T cells having a low GLUT1 expression level, and
c) optionally, isolating the selected T cells having a low GLUT1 expression level.

18. The method according to claim 16, wherein said T cells having a low GLUT1 expression level corresponds to the at most 50%, 40%, 30%, 20%, 10% or 5% fraction with the lowest GLUT1 expression level among the total GLUT1+T cell population.

19. The method according to claim 16, wherein quantifying GLUT1 expression level at the cell surface of the T cells is done by flow cytometry.

20. A method of treating cancer in a subject in need thereof, said method comprising:
selecting T cells with improved anti-cancer activity by the method according to claim 16, and
administering to the subject a therapeutic amount of said T cells with improved anti-cancer activity.

21. A method of selecting T cells with improved anti-cancer activity comprising contacting T cells with a glucose transporter 1 (GLUT1) ligand for selecting T cells with improved anti-cancer activity.

22. The method according to claim 16, wherein said GLUT1 ligand is labeled.

23. The method according to claim 16, wherein said GLUT1 ligand comprises a receptor binding domain (RBD) derived from the soluble part of an envelope glycoprotein of a primate T-lymphotropic virus (PTLV), or comprises an antibody or an antigen-binding fragment thereof.

24. The method according to claim 16, wherein said GLUT1 ligand comprises a receptor binding domain (RBD) derived from the soluble part of an envelope glycoprotein of human T-cell leukemia virus type 1 (HTLV-1), human T-cell leukemia virus type 2 (HTLV-2), human T-cell leukemia virus type 3 (HTLV-3), human T-cell leukemia virus type 4 (HTLV-4), simian T-cell leukemia virus type 1 (STLV-1), simian T-cell leukemia virus type 2 (STLV-2), simian T-cell leukemia virus type 3 (STLV-3), or simian T-cell leukemia virus type 5 (STLV-5).

25. The method according to claim 16, wherein said GLUT1 ligand comprises a receptor binding domain (RBD) derived from the soluble part of an envelope glycoprotein of human T-cell leukemia virus type 2 (HTLV-2).

26. The method according to claim 16, wherein said receptor binding domain (RBD) comprises or consists of the amino acid sequence SEQ ID NO: 15.

27. A method of determining an anti-cancer therapeutic efficacy of T cells, comprising monitoring glucose transporter 1 (GLUT1) as a biomarker of anti-cancer therapeutic efficacy of T cells.

28. The method according to claim 16, wherein said T cells are selected from the group consisting of conventional CD4⁺T cells, conventional CD8⁺T cells, γδ T cells and double negative (DN) T cells.

29. The method according to claim 16, wherein said T cells are chimeric antigen receptor (CAR) T cells.

30. The method according to claim 16, wherein said cancer is a blood cancer or a solid tumor.

31. The method according to claim 20, wherein said GLUT1 ligand comprises a receptor binding domain (RBD) derived from the soluble part of an envelope glycoprotein of human T-cell leukemia virus type 1 (HTLV-1), human T-cell leukemia virus type 2 (HTLV-2), human T-cell leukemia virus type 3 (HTLV-3), human T-cell leukemia virus type 4 (HTLV-4), simian T-cell leukemia virus type 1 (STLV-1), simian T-cell leukemia virus type 2 (STLV-2), simian T-cell leukemia virus type 3 (STLV-3), or simian T-cell leukemia virus type 5 (STLV-5).

32. The method according to claim 20, wherein said receptor binding domain (RBD) comprises or consists of the amino acid sequence SEQ ID NO: 15.

33. The method according to claim 20, wherein said T cells are selected from the group consisting of conventional CD4⁺T cells, conventional CD8⁺T cells, γδ T cells and double negative (DN) T cells.

34. The method according to claim 20, wherein said T cells are chimeric antigen receptor (CAR) T cells.

35. The method according to claim 20, wherein said cancer is a blood cancer or a solid tumor.


SEQUENCE LISTING (52 sequences)

SEQ ID NO: 1 - protein, 492 aa, Homo sapiens
  GLUT1
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 2 - protein, 238 aa, Artificial Sequence
  GFP
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - protein, 228 aa, Artificial Sequence
  Rabbit Fc fragment
APSTCSKPTCPPPELLGGPSVFIFPPKPKDTLMISRTPEVTCVVVDVSQDDPEVQFTWYINNEQVRTARPPLREQQFDCTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 4 - protein, 229 aa, Artificial Sequence
  Mouse Fc fragment
VDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 5 - protein, 312 aa, Artificial Sequence
  HTLV1 SU
MGKFLATLILFFQFCPLILGDYSPSCCTLTIGVSSYHSKPCNPAQPVCSWTLDLLALSADQALQPPCPNLVSYSSYHATYSLYLFPHWIKKPNRNGGGYYSASYSDPCSLKCPYLGCQSWTCPYTGAVSSPYWKFQQDVNFTQEVSRLNINLHFSKCGFPFSLLVDAPGYDPIWFLNTEPSQLPPTAPPLLPHSNLDHILEPSIPWKSKLLTLVQLTLQSTNYTCIVCIDRASLSTWHVLYSPNVSVPSSSSTPLLYPSLALPAPHLTLPFNWTHCFDPQIQAIVSSPCHNSLILPPFSLSPVPTLGSRSRR

SEQ ID NO: 6 - protein, 228 aa, Artificial Sequence
  HTLV1 SU fragment
MGKFLATLILFFQFCPLIFGDYSPSCCTLTIGVSSYHSKPCNPAQPVCSWTLDLLALSADQALQPPCPNLVSYSSYHATYSLYLFPHWTKKPNRNGGGYYSASYSDPCSLKCPYLGCQSWTCPYTGAVSSPYWKFQHDVNFTQEVSRLNINLHFSKCGFPFSLLVDAPGYDPIWFLNTEPSQLPPTAPPLLPHSNLDHILEPSIPWKSKLLTLVQLTLQSTNYTCIVC

SEQ ID NO: 7 - protein, 182 aa, Artificial Sequence
  HTLV1 SU fragment
MGKFLATLILFFQFCPLIFGDYSPSCCTLTIGVSSYHSKPCNPAQPVCSWTLDLLALSADQALQPPCPNLVSYSSYHATYSLYLFPHWTKKPNRNGGGYYSASYSDPCSLKCPYLGCQSWTCPYTGAVSSPYWKFQHDVNFTQEVSRLNINLHFSKCGFPFSLLVDAPGYDPIWFLNTEPSQ

SEQ ID NO: 8 - protein, 51 aa, Artificial Sequence
  HTLV1 SU fragment
IKKPNPNGGGYYLASYSDPCSLKCPYLGCQSWTCPYTGAVSSPYWKFQQDV

SEQ ID NO: 9 - protein, 51 aa, Artificial Sequence
  HTLV1 SU fragment
VKKPNRNGGGYYLASYSDPCSLKCPYLGCQSWTCPYTGAVSSPYWKFQQDV

SEQ ID NO: 10 - protein, 51 aa, Artificial Sequence
  HTLV1 SU fragment
IKKPNRNGGGYYLASYSDPCSLKCPYLGCQSWTCPYTGAVSSPYWKFQQDV

SEQ ID NO: 11 - protein, 51 aa, Artificial Sequence
  HTLV1 SU fragment
IKKPNRNGGGYYLASYSDPCSLKCPYLGCQSWTCPYTGPVSSPYWKFQQDV

SEQ ID NO: 12 - protein, 57 aa, Artificial Sequence
  HTLV1 SU fragment
IKKPNRNGGGYHSASYSDPCSLKCPYLGCQSWTCPYAGAVSSPYWKFQQDVNFTQEV

SEQ ID NO: 13 - protein, 304 aa, Artificial Sequence
  HTLV2 SU
MGNVFFLLLFSLTHFPPVQQSRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQPPPTPPPLVHDSDLEHVLTPSTSWTTKMLKFIQLTLQSTNYSCMVCVDRSSLSSWHVLYTPNISIPQQTSSRTILFPSLALPAPPFQPFPWTHCYQPRLQAITTDDCNNSIILPPFSLAPVPPPAT

SEQ ID NO: 14 - protein, 224 aa, Artificial Sequence
  HTLV2 SU fragment
MGNVFFLLLFSLTHFPLAQQSRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQPPPTSPPLVHDSDLEHVLTPSTSWTTKILKFIQLTLQSTNYSCMVC

SEQ ID NO: 15 - protein, 178 aa, Artificial Sequence
  HTLV2 SU fragment
MGNVFFLLLFSLTHFPLAQQSRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQ

SEQ ID NO: 16 - protein, 51 aa, Artificial Sequence
  HTLV2 SU fragment
IRKPNRQGLGYYSPSYNDPCSLQCPYLGSQSWTCPYTAPVSTPSWNFHSDV

SEQ ID NO: 17 - protein, 315 aa, Artificial Sequence
  HTLV3 SU
MGKSGLYFSLICFYTLFPSSFGNPSRCTLFIGASSYHSDPCGSNHPRCTWRLDLFSLTKDQSLSPPCPGLVTYSQYHKPYSLYVFPHWIAKPDRRGLGYYSASYSDPCAIQCPYLGCQSWTCPYTGPVSNPHWKYTSDLNFTQEVSSISLHLHFSKCGSSFSFLLDAPGYDPVWLLSSQATQIPPTPAPLIQDSDLQHILEPSIPWSSKILNLILLALKSTNYSCMVCVDRSSLSSWHVLYDPLKAPSSPDPQAQSILRPSLAIPASNITPPFPWTHCYRPPLQAISSENCNNSVILPPFSLSPIPDVSRPRKRR

SEQ ID NO: 18 - protein, 180 aa, Artificial Sequence
  HTLV3 SU fragment
MGKSGLYFSLICFYTLFPSSFGNPSRCTLFIGASSYHSDPCGSNHPRCTWRLDLFSLTKDQSLSPPCPGLVTYSQYHKPYSLYVFPHWIAKPDRRGLGYYSASYSDPCAIQCPYLGCQSWTCPYTGPVSNPHWKYTSDLNFTQEVSSISLHLHFSKCGSSFSFLLDAPGYDPVWLLSSQA

SEQ ID NO: 19 - protein, 307 aa, Artificial Sequence
  HTLV4 SU
MGNVLFLTLLATLGIPVLQASRCTITVGISSYHSSPCSPAQPLCTWALDLVSITKDQLLYPPCQNLITYSNYHKTYSLYLFPHWVQKPLRRGLGYYSASYSDPCSLQCPYLGSQSWTCPYTGPVSSPTWRFSTDVNFTQEVSRVSLKLHFSKCGSSLTLLIDAPGYDPLWYLTSEPTQEPPTPPPLVSDSDLEHVLTPSASWASKMLTLIHLTLQSTNYSCMVCIDRASLSSWHVLYTPNISSNAPSKPIVRPSLALSAPRPQPFPWTHCYQPQVQAVTTAKCNNSIILPPFSLSPLPGAPLTRRRR

SEQ ID NO: 20 - protein, 224 aa, Artificial Sequence
  HTLV4 SU fragment
MGNVLFLTLLATLGIPVLQASRCTITVGISSYHSSPCSPAQPLCTWALDLVSITKDQLLYPPCQNLITYSNYHKTYSLYLFPHWVQKPLRRGLGYYSASYSDPCSLQCPYLGSQSWTCPYTGPVSSPTWRFSTDVNFTQEVSRVSLKLHFSKCGSSLTLLIDAPGYDPLWYLTSEPTQEPPTPPPLVSDSDLEHVLTPSASWASKMLTLIHLTLQSTNYSCMVC

SEQ ID NO: 21 - protein, 178 aa, Artificial Sequence
  HTLV4 SU fragment
MGNVLFLTLLATLGIPVLQASRCTITVGISSYHSSPCSPAQPLCTWALDLVSITKDQLLYPPCQNLITYSNYHKTYSLYLFPHWVQKPLRRGLGYYSASYSDPCSLQCPYLGSQSWTCPYTGPVSSPTWRFSTDVNFTQEVSRVSLKLHFSKCGSSLTLLIDAPGYDPLWYLTSEPTQ

SEQ ID NO: 22 - protein, 312 aa, Artificial Sequence
  STLV1 SU
MGKFLATLILFFQFCPLILGDYSPSCCTLTIGVSSYLSKPCNPAQPVCSWTLDLLALSADQALQPPCPNLVSYSSYHATYSLYLFPHWIKKPNRNGGGYYSASYSDPCSLKCPYLGCQSWTCPYTGVVSSPYWKFQQDVNFTQEVSHLNINLHFSKCGFPFSLLIDAPGYDPIWFLNTEPSQLPPTAPPLLPHSNLDHILEPSIPWKSKLLTLVQLTLQSTNYTCIVCIDRASLSTWHVLYSPNVSVPSSSSTPLLYPSLALPAPHLTLPFNWTHCFDPQIQAIVSSPCHNSLILPPFSLSPVPTLGSRSRR

SEQ ID NO: 23 - protein, 179 aa, Artificial Sequence
  STLV1 SU fragment
MGKFLATLILFFQFCPLILGDYSPSCCILTIGVSSYHSKPCNPAQPVCSWTLDLLALSADQALQPPCPNLVSYSSYHATYSLYLFPHWIKKPNRNGGGYYSASYSDPCSLKCPYLGCQSWTCPYTGAVSSPYWKFQQDVNFTQEVSHLNINLHFSKCGFPFSLLIDAPGYDPIWFLNTE

SEQ ID NO: 24 - protein, 308 aa, Artificial Sequence
  STLV2 SU
MGKIIAFLLFHLTCITITKQSRCTLTVGVSSYHSSPCSLAQPICTWDLDLHSLTTDQRLYPPCPNLVSYSNFHKSYSLYLFPHWVKKPNRQGLGYYSASYSDPCSLQCPYLGSQSWTCPYTGPISSPSWRFHRDVNFTQEVNHVTLRLHFSRCGSSMTLLIDAPGYDPLWFISSEPTQPPPTSPPLVRDSDLEHILTPSSSWATRMLTLIQLTLQSTNYSCMVCIDRTSLSSWHVLYTPNISASPGGDSLPILYPSLALPAPQPQPFSWSHCYQPHLQAVTTANCNNSIVLPPFSLTPVPSPGTRSRR

SEQ ID NO: 25 - protein, 175 aa, Artificial Sequence
  STLV2 SU fragment
MGKIIAFLLFHLTCITITKQSRCTLTVGVSSYHSSPCSLAQPICTWDLDLHSLTTDQRLYPPCPNLVSYSNFHKSYSLYLFPHWVKKPNRQGLGYYSASYSDPCSLQCPYLGSQSWTCPYTGPISSPSWRFHRDVNFTQEVNHVTLRLHFSRCGSSMTLLIDAPGYDPLWFISSE

SEQ ID NO: 26 - protein, 310 aa, Artificial Sequence
  STLV3 SU
MGKFGLYCLVHLYILLPASSGNPSRCTLFIGASSYHSSPCGSSLPRCTWNLDLFSLTKDQSLSPPCPDLITYSQYHKPYSLYVFPHWITKPNRRGLGYYSASYSDPCAIQCPYLGCQSWTCPYTGPVSSPHWRYTYDLNFTQEVSSVSLHLHFSKCGSSFSFLLDAPGYDPVWFLSSQATQAPPTPAPLIRDSDLQYILEPPIPWSSKILNLILLTLKSTNYSCMVCVDRSSLSSWHVLYGPTQVPSPPDPQARSILRPALAIPASNITPPFPWTHCYRPPPQAISSENCNNSVVLPPFSLSPIPNVSRP

SEQ ID NO: 27 - protein, 181 aa, Artificial Sequence
  STLV3 SU fragment
MGKSGFYFCFIYTLFPASFGNPSRCTLFIGASSYHSDPCGSNHPQCTWRLDLFSLTRDQSLSPPCPDLVTYSQYHKPYSLYVFPHWMAKPNRQGLGYYSASYSDPCAIQCPYLGCQSWTCPYTGPVSSPHWKYSSDLNFTQEVSSISLHLHFSKCGSSFSFLLDAPGYDPVWFLSSQATQV

SEQ ID NO: 28 - protein, 312 aa, Artificial Sequence
  STLV5 SU
MGKSLFFFCIIVQACLPTLCDRGPSCCTLTVGVSSYHSKPCNPTQPICSWTLDLLTLTTDQALQPPCPNLIGYSNYHATYPLYLFPHWVKKPNRGGGGYYSASYSDPCSLKCPYLGCQSWTCPYTGAVSGPYWKYQTDVNFTQDVSRLTVHLHFSKCGFPFSLLIDAPGYDPIWLINSEPSQLPPTAPPLLPHSNLDHILEPSIPWKSKLLTLVQLTLQSTNYTCIVCLDRASLSTWHVLYSPNMSVPSSPSVPLIYPSLALPAPHLALPFNWTHCFDPKLQAITSAHCYNALILPPFSLSPVPALSHRTRR

SEQ ID NO: 29 - protein, 418 aa, Artificial Sequence
  H2.RBD fused to GFP
MGNVFFLLLFSLTHFPLAQQSRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 30 - protein, 408 aa, Artificial Sequence
  H2.RBD fused to rabbit Fc
MGNVFFLLLFSLTHFPLAQQSRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQGSAPSTCSKPTCPPPELLGGPSVFIFPPKPKDTLMISRTPEVTCVVVDVSQDDPEVQFTWYINNEQVRTARPPLREQQFDCTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 31 - protein, 409 aa, Artificial Sequence
  H2.RBD fused to mouse Fc
MGNVFFLLLFSLTHFPLAQQSRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQGSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 292 aa, Artificial Sequence
  HTLV1 SU w/o peptide signal
DYSPSCCTLTIGVSSYHSKPCNPAQPVCSWTLDLLALSADQALQPPCPNLVSYSSYHATYSLYLFPHWIKKPNRNGGGYYSASYSDPCSLKCPYLGCQSWTCPYTGAVSSPYWKFQQDVNFTQEVSRLNINLHFSKCGFPFSLLVDAPGYDPIWFLNTEPSQLPPTAPPLLPHSNLDHILEPSIPWKSKLLTLVQLTLQSTNYTCIVCIDRASLSTWHVLYSPNVSVPSSSSTPLLYPSLALPAPHLTLPFNWTHCFDPQIQAIVSSPCHNSLILPPFSLSPVPTLGSRSRR

SEQ ID NO: 33 - protein, 208 aa, Artificial Sequence
  HTLV1 SU fragment w/o peptide signal
DYSPSCCTLTIGVSSYHSKPCNPAQPVCSWTLDLLALSADQALQPPCPNLVSYSSYHATYSLYLFPHWTKKPNRNGGGYYSASYSDPCSLKCPYLGCQSWTCPYTGAVSSPYWKFQHDVNFTQEVSRLNINLHFSKCGFPFSLLVDAPGYDPIWFLNTEPSQLPPTAPPLLPHSNLDHILEPSIPWKSKLLTLVQLTLQSTNYTCIVC

SEQ ID NO: 34 - protein, 162 aa, Artificial Sequence
  HTLV1 SU fragment w/o peptide signal
DYSPSCCTLTIGVSSYHSKPCNPAQPVCSWTLDLLALSADQALQPPCPNLVSYSSYHATYSLYLFPHWTKKPNRNGGGYYSASYSDPCSLKCPYLGCQSWTCPYTGAVSSPYWKFQHDVNFTQEVSRLNINLHFSKCGFPFSLLVDAPGYDPIWFLNTEPSQ

SEQ ID NO: 35 - protein, 284 aa, Artificial Sequence
  HTLV2 SU w/o peptide signal
SRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQPPPTPPPLVHDSDLEHVLTPSTSWTTKMLKFIQLTLQSTNYSCMVCVDRSSLSSWHVLYTPNISIPQQTSSRTILFPSLALPAPPFQPFPWTHCYQPRLQAITTDDCNNSIILPPFSLAPVPPPAT

SEQ ID NO: 36 - protein, 204 aa, Artificial Sequence
  HTLV2 SU fragment w/o peptide signal
SRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQPPPTSPPLVHDSDLEHVLTPSTSWTTKILKFIQLTLQSTNYSCMVC

SEQ ID NO: 37 - protein, 158 aa, Artificial Sequence
  HTLV2 SU fragment w/o peptide signal
SRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQ

SEQ ID NO: 38 - protein, 293 aa, Artificial Sequence
  HTLV3 SU w/o peptide signal
NPSRCTLFIGASSYHSDPCGSNHPRCTWRLDLFSLTKDQSLSPPCPGLVTYSQYHKPYSLYVFPHWIAKPDRRGLGYYSASYSDPCAIQCPYLGCQSWTCPYTGPVSNPHWKYTSDLNFTQEVSSISLHLHFSKCGSSFSFLLDAPGYDPVWLLSSQATQIPPTPAPLIQDSDLQHILEPSIPWSSKILNLILLALKSTNYSCMVCVDRSSLSSWHVLYDPLKAPSSPDPQAQSILRPSLAIPASNITPPFPWTHCYRPPLQAISSENCNNSVILPPFSLSPIPDVSRPRKRR

SEQ ID NO: 39 - protein, 158 aa, Artificial Sequence
  HTLV3 SU fragment w/o peptide signal
NPSRCTLFIGASSYHSDPCGSNHPRCTWRLDLFSLTKDQSLSPPCPGLVTYSQYHKPYSLYVFPHWIAKPDRRGLGYYSASYSDPCAIQCPYLGCQSWTCPYTGPVSNPHWKYTSDLNFTQEVSSISLHLHFSKCGSSFSFLLDAPGYDPVWLLSSQA

SEQ ID NO: 40 - protein, 287 aa, Artificial Sequence
  HTLV4 SU w/o peptide signal
SRCTITVGISSYHSSPCSPAQPLCTWALDLVSITKDQLLYPPCQNLITYSNYHKTYSLYLFPHWVQKPLRRGLGYYSASYSDPCSLQCPYLGSQSWTCPYTGPVSSPTWRFSTDVNFTQEVSRVSLKLHFSKCGSSLTLLIDAPGYDPLWYLTSEPTQEPPTPPPLVSDSDLEHVLTPSASWASKMLTLIHLTLQSTNYSCMVCIDRASLSSWHVLYTPNISSNAPSKPIVRPSLALSAPRPQPFPWTHCYQPQVQAVTTAKCNNSIILPPFSLSPLPGAPLTRRRR

SEQ ID NO: 41 - protein, 204 aa, Artificial Sequence
  HTLV4 SU fragment w/o peptide signal
SRCTITVGISSYHSSPCSPAQPLCTWALDLVSITKDQLLYPPCQNLITYSNYHKTYSLYLFPHWVQKPLRRGLGYYSASYSDPCSLQCPYLGSQSWTCPYTGPVSSPTWRFSTDVNFTQEVSRVSLKLHFSKCGSSLTLLIDAPGYDPLWYLTSEPTQEPPTPPPLVSDSDLEHVLTPSASWASKMLTLIHLTLQSTNYSCMVC

SEQ ID NO: 42 - protein, 158 aa, Artificial Sequence
  HTLV4 SU fragment w/o peptide signal
SRCTITVGISSYHSSPCSPAQPLCTWALDLVSITKDQLLYPPCQNLITYSNYHKTYSLYLFPHWVQKPLRRGLGYYSASYSDPCSLQCPYLGSQSWTCPYTGPVSSPTWRFSTDVNFTQEVSRVSLKLHFSKCGSSLTLLIDAPGYDPLWYLTSEPTQ

SEQ ID NO: 43 - protein, 292 aa, Artificial Sequence
  STLV1 SU w/o peptide signal
DYSPSCCTLTIGVSSYLSKPCNPAQPVCSWTLDLLALSADQALQPPCPNLVSYSSYHATYSLYLFPHWIKKPNRNGGGYYSASYSDPCSLKCPYLGCQSWTCPYTGVVSSPYWKFQQDVNFTQEVSHLNINLHFSKCGFPFSLLIDAPGYDPIWFLNTEPSQLPPTAPPLLPHSNLDHILEPSIPWKSKLLTLVQLTLQSTNYTCIVCIDRASLSTWHVLYSPNVSVPSSSSTPLLYPSLALPAPHLTLPFNWTHCFDPQIQAIVSSPCHNSLILPPFSLSPVPTLGSRSRR

SEQ ID NO: 44 - protein, 159 aa, Artificial Sequence
  STLV1 SU fragment w/o peptide signal
DYSPSCCILTIGVSSYHSKPCNPAQPVCSWTLDLLALSADQALQPPCPNLVSYSSYHATYSLYLFPHWIKKPNRNGGGYYSASYSDPCSLKCPYLGCQSWTCPYTGAVSSPYWKFQQDVNFTQEVSHLNINLHFSKCGFPFSLLIDAPGYDPIWFLNTE

SEQ ID NO: 45 - protein, 288 aa, Artificial Sequence
  STLV2 SU w/o peptide signal
SRCTLTVGVSSYHSSPCSLAQPICTWDLDLHSLTTDQRLYPPCPNLVSYSNFHKSYSLYLFPHWVKKPNRQGLGYYSASYSDPCSLQCPYLGSQSWTCPYTGPISSPSWRFHRDVNFTQEVNHVTLRLHFSRCGSSMTLLIDAPGYDPLWFISSEPTQPPPTSPPLVRDSDLEHILTPSSSWATRMLTLIQLTLQSTNYSCMVCIDRTSLSSWHVLYTPNISASPGGDSLPILYPSLALPAPQPQPFSWSHCYQPHLQAVTTANCNNSIVLPPFSLTPVPSPGTRSRR

SEQ ID NO: 46 - protein, 155 aa, Artificial Sequence
  STLV2 SU fragment w/o peptide signal
SRCTLTVGVSSYHSSPCSLAQPICTWDLDLHSLTTDQRLYPPCPNLVSYSNFHKSYSLYLFPHWVKKPNRQGLGYYSASYSDPCSLQCPYLGSQSWTCPYTGPISSPSWRFHRDVNFTQEVNHVTLRLHFSRCGSSMTLLIDAPGYDPLWFISSE

SEQ ID NO: 47 - protein, 289 aa, Artificial Sequence
  STLV3 SU w/o peptide signal
NPSRCTLFIGASSYHSSPCGSSLPRCTWNLDLFSLTKDQSLSPPCPDLITYSQYHKPYSLYVFPHWITKPNRRGLGYYSASYSDPCAIQCPYLGCQSWTCPYTGPVSSPHWRYTYDLNFTQEVSSVSLHLHFSKCGSSFSFLLDAPGYDPVWFLSSQATQAPPTPAPLIRDSDLQYILEPPIPWSSKILNLILLTLKSTNYSCMVCVDRSSLSSWHVLYGPTQVPSPPDPQARSILRPALAIPASNITPPFPWTHCYRPPPQAISSENCNNSVVLPPFSLSPIPNVSRP

SEQ ID NO: 48 - protein, 161 aa, Artificial Sequence
  STLV3 SU fragment w/o peptide signal
NPSRCTLFIGASSYHSDPCGSNHPQCTWRLDLFSLTRDQSLSPPCPDLVTYSQYHKPYSLYVFPHWMAKPNRQGLGYYSASYSDPCAIQCPYLGCQSWTCPYTGPVSSPHWKYSSDLNFTQEVSSISLHLHFSKCGSSFSFLLDAPGYDPVWFLSSQATQV

SEQ ID NO: 49 - protein, 293 aa, Artificial Sequence
  STLV5 SU w/o peptide signal
CDRGPSCCTLTVGVSSYHSKPCNPTQPICSWTLDLLTLTTDQALQPPCPNLIGYSNYHATYPLYLFPHWVKKPNRGGGGYYSASYSDPCSLKCPYLGCQSWTCPYTGAVSGPYWKYQTDVNFTQDVSRLTVHLHFSKCGFPFSLLIDAPGYDPIWLINSEPSQLPPTAPPLLPHSNLDHILEPSIPWKSKLLTLVQLTLQSTNYTCIVCLDRASLSTWHVLYSPNMSVPSSPSVPLIYPSLALPAPHLALPFNWTHCFDPKLQAITSAHCYNALILPPFSLSPVPALSHRTRR

SEQ ID NO: 50 - protein, 398 aa, Artificial Sequence
  H2.RBD fused to GFP w/o peptide signal
SRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 51 - protein, 388 aa, Artificial Sequence
  H2.RBD fused to rabbit Fc w/o peptide signal
SRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQGSAPSTCSKPTCPPPELLGGPSVFIFPPKPKDTLMISRTPEVTCVVVDVSQDDPEVQFTWYINNEQVRTARPPLREQQFDCTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 52 - protein, 389 aa, Artificial Sequence
  H2.RBD fused to mouse Fc w/o peptide signal
SRCTLTVGISSYHSSPCSPTQPVCTWNLDLNSLTTDQRLHPPCPNLITYSGFHKTYSLYLFPHWIKKPNRQGLGYYSPSYNDPCSLQCPYLGCQSWTCPYTGPVSSPSWKFHSDVNFTQEVSQVSLRLHFSKCGSSMTLLVDAPGYDPLWFITSEPTQGSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK